(19) United States
(12) Patent Application Publication
(10) Pub. No.: US 2026/0237068 A1
(43) Pub. Date: Aug. 13, 2026

(54) IMAGE ANALYSIS SYSTEM FOR OTOSCOPY IMAGES

(71) Applicant: Tympa Health Technologies Limited, London (GB)

(72) Inventors: Krishan RAMDOO, London (GB); John Robert MADDISON, London (GB); Juan Pablo STOCCA, London (GB); Almpion RATSAKOU, London (GB); Toby Charlie Andrew EVANS, London (GB)

(73) Assignee: Tympa Health Technologies Limited, London (GB)

(21) Appl. No.: 19/632,120

(22) Filed: Mar. 27, 2026

Related U.S. Application Data
(63) Continuation of application No. PCT/GB2025/050410, filed on Feb. 28, 2025.

(30) Foreign Application Priority Data
Feb. 29, 2024 (EP) 24386021.0
Mar. 8, 2024 (GB) 2403367.2

(51) Int. Cl. G06T 7/00; A61B 1/00; A61B 1/227; A61B 5/00; G06V 10/764; G06V 10/82; G16H 50/20

(52) U.S. Cl. CPC G06T 7/0012; A61B 1/000094; A61B 1/000096; A61B 1/227; A61B 5/004; A61B 5/7267; G06V 10/764; G06V 10/82; G16H 50/20; G06T 2200/24; G06T 2207/10016; G06T 2207/10068; G06T 2207/20081; G06T 2207/20084; G06V 2201/03

(57) ABSTRACT

The present disclosure relates to an otoscopy application for a mobile device for processing otoscopy image data. The otoscopy application acquires image data of a subject's ear canal using a camera of the mobile device. An image analysis module is configured to process the image data using a trained machine learning model stored at the mobile device, wherein the trained machine learning model is configured to generate classification data for the image data, the classification data distinguishing between at least a normal classification indicating the image data is representative of a healthy ear and one or more abnormal classifications relating to abnormal conditions of the ear. An upload module transmits the image data and the classification data over the network to a remote review system for review.

### CROSS-REFERENCE TO RELATED APPLICATION

This application claims the benefit under 35 U.S.C. § 120 as a continuation of PCT Patent Application No. PCT/GB2025/050410, filed on Feb. 28, 2025, which claims priority to European Patent Convention application Ser. No. 24/386,021.0, filed Feb. 29, 2024, and United Kingdom Application No. 2403367.2, filed Mar. 8, 2024, the entire contents of which are incorporated herein by reference.

### FIELD OF THE PRESENT DISCLOSURE

The present disclosure relates to computer-implemented methods and systems for analyzing digital images of the ear canal such as those acquired using an otoscope.

### BACKGROUND

Otoscopy is a medical examination technique that involves visually inspecting the ear canal and eardrum using an otoscope. It can help health professionals to diagnose conditions relating to the ear, which can prevent conditions worsening to the point where the physical symptoms extend beyond the ear canal.

Present systems exist that may allow imaging during otoscopy to obtain photos or videos of the ear canal, such as by using an otoscope attachment for a smartphone. While such devices simplify examination for ear care practitioners and other health professionals and make otoscopy available to a wider range of users with comparatively simple equipment and limited training requirements, interpretation of otoscopy images may be challenging for users with limited experience.

### SUMMARY

The present disclosure describes computer-implemented and/or device-implemented methods and systems configured to interpret, classify, and/or annotate otoscopy images to e.g., facilitate review of otoscopy images by users with limited experiences. The computer-implemented and/or device implemented methods and systems may be configured to classify one or more images and/or one or more portions of video of a subject's ear canal, tympanic membrane, and/or other ear anatomical features, as healthy, abnormal, or as containing ear wax, as described elsewhere herein. The abnormal conditions, as described elsewhere herein, may refer to pathological or disease conditions. Providing on device, on computer-implemented, and/or on cloud-base (e.g., networked remote computer-based servers) classification, as described elsewhere herein, may allow diagnostic support to be provided to a health care practitioner immediately during an otoscopy appointment, allowing more efficient decision-making, escalation (of intervention(s)), etc. The disclosure describes computer-implemented and/or device-implemented methods and systems configured to upload image data and classification data to a remote review system provide experienced users (e.g., ear nose and throat specialists and audiologist) to review the machine learning and/or artificial intelligence derived classifications to ensure appropriate and timely follow-up. This approach can also enable detection of problems in a setting where diagnostics may not conventionally be performed, e.g., during wax removal appointments or simple otoscopy of the ear canal or drum in non-specialist settings, allowing for early intervention.

In an aspect, the disclosure describes a computer readable medium storing software code for implementing, when run on a mobile and/or other computing device, an otoscopy application for processing otoscopy image data, the otoscopy application comprising: an imaging module configured to receive image data of a subject's ear canal, receiving such image data by using a camera system of the computing device to acquire such images; an image analysis module configured to process the image data using a trained machine learning model, which may be stored at the mobile or other computing device (e.g., a remote server and/or a cloud based server, described elsewhere herein) or a second computing device in communication therewith, wherein the trained machine learning model is configured to generate classification data for the image data, the classification data distinguishing between at least a normal classification, indicating that the image data is representative of a healthy ear, and one or more abnormal classifications relating to abnormal conditions of the ear; and an upload module configured to transmit the image data and the classification data over a network to a remote review system.

In some embodiments, the machine learning model comprises: a first classifier adapted to output an initial classification distinguishing between at least normal (e.g., healthy) and abnormal classification of one or more images and/or video segments of a subject's ear; and a second classifier adapted to output one of a plurality of diagnostic classifications corresponding to respective abnormal conditions (e.g., pathological or disease conditions), wherein the image analysis module is configured to apply the first classifier to the image data (e.g., to obtain a diagnostic classification of healthy, abnormal, or wax in an image, video, or portion thereof or to obtain a recommendation for action (i.e., recommended treatment)), and to apply the second classifier in response to the first classifier classifying the image data (e.g., as abnormal), to obtain a diagnostic classification of and/or recommendation for action for the subject. In some embodiments, the first classifier, the second classifier, or a combination thereof, may comprise a neural network, e.g., a neural network trained on one or more images and/or one or more segments of video with corresponding classification labels. In some embodiments, the first classifier outputs a classification selected from a set containing at least: a normal class indicating a healthy ear, an abnormal class indicating that an abnormal condition is present, and a wax class indicating that the image data indicates the presence of wax in the ear canal. In some embodiments, the second classifier outputs a classification selected from a set containing at least: a plurality of diagnostic classes for respective individual diagnostic conditions (e.g., the classes set out in Table 2, described elsewhere herein), and a generic abnormal classification for abnormalities (e.g., an abnormality outside of the plurality of individual diagnostic classes).

In some embodiments, the application is further configured to determine a referral indication for the image data in dependence on the classification data to indicate whether the image data should be referred for review. In some embodiments, the referral indication indicates referral if the image data is classified (e.g., by the first classifier) as abnormal and non-referral otherwise. In some embodiments, the transmitted data comprises the referral indication.

In some embodiments, the application is further configured to determine a recommendation indication in dependence on the classification data to indicate whether the subject should receive a recommendation for action. In some embodiments, the recommendation indication indicates recommendation if the image data is classified as abnormal and non-recommendation otherwise. In some embodiments, the transmitted data includes the recommendation indication.

In some embodiments, the application is further configured to determine a recommendation indication in dependence on the referral indication to indicate whether the subject should receive a recommendation for action. In some embodiments, the recommendation indication indicates recommendation if the referral indication indicates referral and non-recommendation otherwise. In some embodiments, the transmitted data includes the recommendation indication.

In some embodiments, the recommendation for action is for action taken by a subject, a healthcare provider (i.e., audiology practitioner or other medical professional or paraprofessional), or both. The recommendation for action may be for action taken by the subject. The recommendation for action may be for action taken by the healthcare provider. The recommendation for action may be for action taken by the subject and the healthcare provider.

In some embodiments, the recommendation for action is a treatment recommendation. The treatment recommendation may indicate a course of action related to the diagnostic classification. The treatment recommendation may inform the subject, the healthcare provider, or both regarding effective treatment options related to the diagnostic classification.

In some embodiments, the treatment recommendation is a patient group directive (or equivalent, i.e., standing order, collaborative practice agreement, medication protocol, medicinal product directive, or treatment protocol). In some embodiments, the recommendation for action is a patient group directive or other treatment recommendation. In some embodiments, the treatment recommendation is a patient group directive, clinical practice guideline, pharmacological guideline, preventative recommendation, alternative recommendation, complementary recommendation, nutritional guideline, or rehabilitative guideline.

In some embodiments, the transmitted data further comprises subject data and/or appointment data relating to the subject (e.g., acquired via user input into the application).

In some embodiments, the image data comprises at least one of: one or more images; and video data (e.g., a video clip, portion, and/or segment thereof), wherein the application is configured to apply the machine learning model to one or more frames of the video data.

In some embodiments, the application configured to: apply an image classifier (e.g., the first and/or second classifier, described elsewhere herein) to a series of frames of video to obtain a time series of classification values corresponding to respective frames of the video; and apply a smoothing operation to the time series of classification values to obtain classifications for individual frames, a plurality of frames, and/or groups of frames. Such smoothening may reduce the impact of outliers and improve classification performance. In some embodiments, the smoothing operation comprises a window function (e.g., a median window function or other averaging window function) applied to successive windows of the classification values, wherein the smoothing operation determines a representative value for each window that is used as the classification value for the frames in the window. In some embodiments, successive windows may be overlapping windows or non-overlapping windows.

In some embodiments, the application is configured to: classify frames of a video clip as normal or abnormal using a first classifier and determine a classification of the video clip as normal or abnormal based on the classifications of the frames, wherein the video clip is classified as abnormal if any frame of the video clip was classified as abnormal. In some embodiments, the application is configured to classify frames of the video clip in accordance with a plurality of diagnostic classes using a second classifier and determine a representative diagnostic classification of the video clip based on the diagnostic classes assigned to the frames. In some embodiments, the representative diagnostic classification corresponds to a majority classification of a set of classifications determined for the frames. In some embodiments, only classifications assigned a class probability by the second classifier that meets a probability threshold are used in determining the representative or majority classification. This can allow inconsistent classification in a video clip to be corrected. In some embodiments, the first and second classifiers correspond to the first and second classifiers described elsewhere herein, also referred to as stage 1 and stage 2 classifiers elsewhere herein.

In some embodiments, the application is configured to include location data in the transmitted classification data indicating one or more locations in the video where a frame was classified as abnormal or as associated with a diagnostic classification. The location data may e.g., indicate a frame or time index in the video of the abnormal frame. The location data may enable efficient review at the review system, described elsewhere herein, allowing a reviewer to navigate directly to the relevant abnormal frames.

In some embodiments, the machine learning model comprises one or more neural networks implementing one or more image classifiers. In some embodiments, the machine learning model implements the first and/or second classifiers, described elsewhere herein. In some embodiments, the machine learning model comprises a neural network (e.g., implementing the first and/or second classifier, described elsewhere herein) which includes: a feature extraction subnetwork configured to receive a representation of an input image and output a plurality of features derived from the input image; a dropout layer configured to receive inputs based on outputs from the feature extraction subnetwork, wherein the dropout layer is arranged to selectively deactivate a proportion of the inputs in accordance with a dropout rate; and a dense layer for generating classification probabilities for each of a set of classifications based on an output of the dropout layer. In some embodiments, the feature extraction subnetwork comprises a convolution layer. In some embodiments, the extraction subnetwork comprises an average pooling layer operating on outputs of the convolution layer. The convolution layer may allow more effective model learning and can enable transfer learning whereby the model training process makes use of a pre-trained convolution layer. The dropout layer may allow overfitting to be counteracted. In some embodiments, the dropout rate may comprise at least about 80%, at least about 90%, or at least about 95%.

In some embodiments, the application is configured to select one or more images or video clips to be uploaded to a remote review system for review by a reviewing user in dependence on the classification data generated for the one or more images or video clips by the machine learning model or based on a referral indication generated in dependence on the classification data. In some embodiments, the application is configured to upload one or more images or video clips in response to one or more images or video clips being classified as abnormal by the machine learning model (or having a referral indication associated with it that was generated based on classification data. In some embodiments, one or more pictures and/or one or more segments or clips of video flagged and/or tagged as abnormal and/or requiring further review may be uploaded. In some embodiments, the application comprises a user interface configured to display acquired image and/or video data, wherein the application is configured to display an indication on the user interface to indicate that displayed image and/or video clip data has been classified as abnormal by the machine learning model.

In some embodiments, the application is configured, in response to a user command to: capture and/or acquire the one or more images and display the one or more images on the user interface; apply the machine learning model to one or more images; and in response to obtaining an abnormal classification for the one or more images, display an indication of a potential detected abnormality on the user interface.

In some embodiments, the application is configured, in response to a user command to: acquire and/or commence recording of one or more video clips and display the one or more video clips on the user interface as it is being recorded; applying the machine learning model to one or more frames of the one or more video clips; and in response to obtaining an abnormal classification for a frame of the one or more video clips, display an indication of a potential detected abnormality on the user interface. In some embodiments, the application is configured to maintain the displayed indication for a predetermined duration after detection of an abnormal frame or after completion of acquiring the one or more images and/or one or more video clips, followed by removing the indication. In some embodiments, the application is configured to display an indication on the user interface during acquisition of image data to indicate that the acquired one or more images and/or one or more video clips are being processed by the machine learning model e.g., to indicate when the machine learning model is active and is applied to one or more frames of the video while recording the video. In some embodiments, the machine learning model may be activated during one or more imaging modes, as described elsewhere herein. In some embodiments, the upload module, and the step of transmitting data to a remote review server may be omitted, and the classification data is outputted to a local device (e.g., a personal computing device and/or mobile device) via an interface of the application, e.g., using user interface features, described elsewhere herein.

In some embodiments, the application is further configured to determine a recommendation indication in dependence on the classification data to indicate whether the subject should receive a recommendation for action. In some embodiments, the recommendation indication indicates recommendation if the image data is classified as abnormal and non-recommendation otherwise. In some embodiments, the transmitted data includes the recommendation indication.

In some embodiments, the application is further configured to determine a recommendation indication in dependence on the referral indication to indicate whether the subject should receive a recommendation for action. In some embodiments, the recommendation indication indicates recommendation if the referral indication indicates referral and non-recommendation otherwise. In some embodiments, the transmitted data includes the recommendation indication.

In some embodiments, the recommendation for action is a treatment recommendation. In some embodiments, the treatment recommendation is a patient group directive. In some embodiments, the recommendation for action is a patient group directive or other treatment recommendation. In some embodiments, the treatment recommendation is a patient group directive, clinical practice guideline, treatment protocol, pharmacological guideline, preventative recommendation, alternative recommendation, complementary recommendation, nutritional guideline, or rehabilitative guideline.

Aspects of the disclosure describe a mobile device comprising a camera and a computer readable medium storing the application, described elsewhere herein. In some embodiments, the mobile device is coupled to an otoscope attachment for the, wherein the attachment comprises a speculum for projecting an image from the speculum onto the camera of the mobile device. In some embodiments, the machine learning model, described elsewhere herein, is stored and/or applied at the mobile device. In some embodiments, the machine learning model is stored and/or applied at a different device and/or system than that of the mobile device.

Aspects of the present disclosure describe a computer-implemented method for processing otoscopy image data. In some embodiments, processing is conducted at the mobile device and/or at a server. In some embodiments, the computer-implemented method comprises: receiving image data of a subject's ear canal; processing the image data using a trained machine learning model, wherein the trained machine learning model comprises: a first classifier adapted to output an initial classification distinguishing between at least a normal classification indicating that the image data is representative of a healthy ear and an abnormal classification indicative of presence of an abnormal condition of the ear; and a second classifier adapted to output one of a plurality of diagnostic classifications corresponding to respective abnormal condition(s), wherein processing comprises: applying the first classifier to the image data to obtain a first classification result, and applying the second classifier in response to the first classifier classifying the image data as abnormal to obtain a second classification result indicating a diagnostic classification for the image data. In some embodiments, the method further comprises outputting the first and second classification results. In some embodiments, the method comprises steps, features, and/or operations as performed by the application, described elsewhere herein.

In some embodiments, the first classifier outputs a classification selected from a set containing at least: a normal class indicating a healthy ear, an abnormal class indicating that an abnormal condition is present, and a wax class indicating that the image data indicates the presence of wax in the ear canal. In some embodiments, the second classifier outputs a classification selected from a set containing at least: a plurality of diagnostic classes for respective individual diagnostic conditions (e.g., pathological or disease conditions), and a generic abnormal classification for abnormalities not covered by the plurality of individual diagnostic classes. In some embodiments, the image data comprises video.

In some embodiments, the method further comprises applying the first and/or second classifiers to one or more frames of the video. In some embodiments, the method further comprises applying the first and/or the second classifier to a series of frames of the video to obtain a time series of classification values corresponding to respective frames of the video; and applying a smoothing operation to the time series of classification values to obtain classifications for individual frames, a plurality of frames, and/or groups of frames. In some embodiments, the smoothing operation comprises a window function applied to successive windows of the classification values. In some embodiments, the window comprises a median or averaging window function. In some embodiments, the first and/or second classifier comprises a trained neural network, as described elsewhere herein. In some embodiments, the method further comprises any of the further steps, features, and/or or operations as performed by the application embodied in the computer readable medium, described elsewhere herein.

Aspects of the disclosure describe a computer-implemented method of processing otoscopy image data, comprising: receiving, at a server system, otoscopy data including a plurality of media items from otoscopy applications at a plurality of mobile devices, and classification data determined for the media items by the applications using a classification model; displaying to a reviewing user via a review application interface, one or more media items with the associated classification data; receiving input from the reviewing user to assign one or more revised classifications to the one or more media items; and associating the revised classifications with the media items. In some embodiments, the classification data comprises normal classifications, abnormal classifications, or a combination thereof (i.e., stage 1 as described elsewhere herein), and/or diagnostic classifications (i.e., stage 2 as described elsewhere herein). In some cases, the classification data comprises review indications indicating that media items have been flagged for review. In some embodiments, the reviewing user may amend stage 1, stage 2, or a combination thereof, and/or the reviewing user may modify the review indication(s) to include or exclude a media item from the review process.

In some embodiments, the method further comprises adding received groups of media items associated with an appointment of a subject to a review queue and making the groups of media items in the queue available to reviewing users for selection; and preforming the steps of displaying and receiving input responsive to selection of a media item or a media item group by a reviewing user in the review application interface. In some embodiments, the media items comprise a video clip, wherein the classification data includes information indicating an abnormal classification assigned to a frame of the video clip and location information indicating a location in the video clip of the frame to which the classification was assigned.

In some embodiments, the method further comprises: displaying the video clip on a playback interface of the review application; and providing a user interface element arranged to initiate playback at a playback location determined in dependence on the location associated with the abnormal classification. In some embodiments, the user interface element comprises a marker element (e.g., a graphical icon) associated with a timeline of the playback interface, the marker element marking the location in the video clip of the frame comprising the abnormal classification on the timeline. In some embodiments, the method comprises moving, responsive to a user interacting with the marker element, a playback position of the video to the location or to a point in the video preceding the location by a predetermined lead-in time and/or to the start of a time region containing the abnormal frame(s). In some embodiments, the method comprises providing a plurality of marker elements indicating respective video clip locations corresponding to respective frames classified as abnormal.

Aspects of the present disclosure describe a system comprising a computer device having a processor with associated memory for performing the methods, as described elsewhere herein. In some embodiments, the system comprises a computer readable medium comprising software (e.g., software code) adapted, when executed by a data processing system (e.g., a processor of the system), to perform the methods, described elsewhere herein. In some embodiments, the method comprises: receiving, at a server system, otoscopy data including a plurality of media items from otoscopy applications at a plurality of mobile devices, and classification data determined for the media items by the applications using a classification model; displaying to a reviewing user via a review application interface, one or more media items with the associated classification data; receiving input from the reviewing user to assign one or more revised classifications to the one or more media items; and associating the revised classifications with the media items. In some embodiments, the media items comprise a video clip, wherein the classification data includes information indicating an abnormal classification assigned to a frame of the video clip and location information indicating a location in the video clip of the frame to which the classification was assigned.

In some embodiments, the method further comprises: displaying the video clip on a playback interface of the review application; and providing a user interface element arranged to initiate playback at a playback location determined in dependence on the location associated with the abnormal classification. In some embodiments, the user interface element comprises a marker element associated with a timeline of the playback interface, the marker element marking the location in the video clip of the frame having the abnormal classification on the timeline, the method comprising moving, responsive to a user interacting with the marker element, a playback position of the video to the location or to a point in the video preceding the location by a predetermined lead-in time. In some embodiments, the method comprises providing a plurality of marker elements indicating respective video clip locations corresponding to respective frames classified as abnormal.

In some embodiments, the computer-implemented method comprises: receiving image receiving image data of a subject's ear canal; processing the image data using a trained machine learning model, wherein the trained machine learning model comprises: a first classifier adapted to output an initial classification distinguishing between at least a normal classification indicating that the image data is representative of a healthy ear and an abnormal classification indicative of presence of an abnormal condition of the ear; and a second classifier adapted to output one of a plurality of diagnostic classifications corresponding to respective abnormal condition, wherein the processing step comprises: applying the first classifier to the image data to obtain a first classification result, and applying the second classifier in response to the first classifier classifying the image data as abnormal to obtain a second classification result indicating a diagnostic classification for the image data, wherein the method further comprises outputting the first and second classification results. In some embodiments, the first classifier outputs a classification selected from a set containing at least: a normal class indicating a healthy ear, an abnormal class indicating that an abnormal condition is present, and a wax class indicating that the image data indicates the presence of wax in the ear canal; and/or the second classifier outputs a classification selected from a set containing at least: a plurality of diagnostic classes for respective individual diagnostic conditions, and a generic abnormal classification for abnormalities not covered by the plurality of individual diagnostic classes. In some embodiments, alternatively or in combination with the plurality of diagnostic classifications, the second classifier comprises a recommendation for action.

In some embodiments, the image data comprises video, wherein the method further comprises applying the first and second classifiers to frames of the video, comprising: applying the first and/or the second classifier to a series of frames of the video to obtain a time series of classification values corresponding to respective frames of the video; and applying a smoothing operation to the time series of classification values to obtain classifications for individual frames, a plurality of frames, and/or groups of frames, wherein the smoothing operation comprises a window function applied to successive windows of the classification values, and wherein the window comprises a median or averaging window function. In some embodiments, the first and/or second classifier comprises a trained neural network. In some embodiments, the method further comprises any of the further steps, features or operations as performed by the application embodied in the computer readable medium, described elsewhere herein.

Aspects of the present disclosure describe a computer program or computer readable medium comprising software code adapted, when executed by a data processing system, to perform a method. In some embodiments, the method comprises: receiving, at a server system, otoscopy data including a plurality of media items from otoscopy applications at a plurality of mobile devices, and classification data determined for the media items by the applications using a classification model; displaying to a reviewing user via a review application interface, one or more media items with the associated classification data; receiving input from the reviewing user to assign one or more revised classifications to the one or more media items; and associating the revised classifications with the media items. In some embodiments, the media items comprise a video clip, wherein the classification data includes information indicating an abnormal classification assigned to a frame of the video clip and location information indicating a location in the video clip of the frame to which the classification was assigned. In some embodiments, the method further comprises displaying the video clip on a playback interface of the review application; and providing a user interface element arranged to initiate playback at a playback location determined in dependence on the location associated with the abnormal classification.

In some embodiments, the user interface element comprises a marker element associated with a timeline of the playback interface, the marker element marking the location in the video clip of the frame having the abnormal classification on the timeline, the method comprising moving, responsive to a user interacting with the marker element, a playback position of the video to the location or to a point in the video preceding the location by a predetermined lead-in time.

In some embodiments, the method comprises providing a plurality of marker elements indicating respective video clip locations corresponding to respective frames classified as abnormal. In some embodiments, the computer-implemented method comprises: receiving image receiving image data of a subject's ear canal; processing the image data using a trained machine learning model, wherein the trained machine learning model comprises: a first classifier adapted to output an initial classification distinguishing between at least a normal classification indicating that the image data is representative of a healthy ear and an abnormal classification indicative of presence of an abnormal condition of the ear; and a second classifier adapted to output one of a plurality of diagnostic classifications corresponding to respective abnormal condition, wherein the processing step comprises: applying the first classifier to the image data to obtain a first classification result, and applying the second classifier in response to the first classifier classifying the image data as abnormal to obtain a second classification result indicating a diagnostic classification for the image data, wherein the method further comprises outputting the first and second classification results. In some embodiments, the first classifier outputs a classification selected from a set containing at least: a normal class indicating a healthy ear, an abnormal class indicating that an abnormal condition is present, and a wax class indicating that the image data indicates the presence of wax in the ear canal; and/or the second classifier outputs a classification selected from a set containing at least: a plurality of diagnostic classes for respective individual diagnostic conditions, and a generic abnormal classification for abnormalities not covered by the plurality of individual diagnostic classes.

In some embodiments, the image data comprises video, wherein the method further comprises applying the first and second classifiers to frames of the vide, comprising: applying the first and/or the second classifier to a series of frames of the video to obtain a time series of classification values corresponding to respective frames of the video; and applying a smoothing operation to the time series of classification values to obtain classifications for individual frames, a plurality of frames, and/or groups of frames, wherein the smoothing operation comprises a window function applied to successive windows of the classification values, and wherein the window comprises a median or averaging window function. In some embodiments, the first and/or second classifier comprises a trained neural network. In some embodiments, the method further comprises any of the further steps, features or operations as performed by the application embodied in the computer readable medium, described elsewhere herein. Aspects of the present disclosure describe a method for identifying a physiological state or condition of an ear of a subject, comprising using a camera-enabled electronic device to capture an image or video from the ear of the subject, and computer processing the image or video to identify the physiological state or condition of the ear of the subject at an accuracy of at least 80%. In some embodiments, the image or video may comprise one or more images and/or one or more segments of video. In some embodiments, the computer processing comprises using a trained machine learning (ML) algorithm to process one or more image or video. In some embodiments, the trained ML algorithm is stored on the camera-enabled electronic device. In some embodiments, the trained ML algorithm is stored on a computer system separate from the camera-enabled electronic device. In some embodiments, the computer system comprises a cloud-based computer system. In some embodiments, the processing of the image or video is conducted with a machine learning algorithm.

Aspects of the present disclosure describe a method of training a machine learning algorithm, comprising: receiving a dataset of one or more images, one or more video segments, or a combination thereof, of an ear of a subject and corresponding physiologic state or condition label of the one or more images, the one or more video segments, or a combination thereof; transforming the dataset to scale and/or resize the dataset; preparing a training data set and a validation data set from the transformed dataset; and training the machine learning algorithm with the training data set and the validation data set, wherein the trained machine learning algorithm has an accuracy of at least about 80% when predicting a physiologic state or condition of one or more images, one or more video segments, or a combination thereof, of a subject's ear.

In some embodiments, the machine learning algorithm comprises a neural network. In some embodiments, the machine learning algorithm comprises a first classifier and a second classifier. In some embodiments, the first classifier is configured to classify a subject's one or more images, one or more video segments, or a combination thereof, as abnormal, normal, or wax. In some embodiments, the second classifier is configured to classify a subject's one or more images, one or more video segments, or a combination thereof, as abnormal, myringosclerosis, otitis externa, perforation, retraction, or trauma.

Aspects of the present disclosure describe a method for identifying a physiological state or condition of an ear of a subject, comprising: receiving an image or video of the ear of the subject; processing the image or video of the ear of the subject with a first machine learning classifier and a second machine learning classifier; and identifying the physiological state or condition of the ear of the subject based on at least an output of the first machine learning classifier, an output of the second machine learning classifier, or a combination thereof.

In some embodiments, the first machine learning classifier, the second machine learning classifier, or a combination thereof, is stored on a camera-enabled electronic device. In some embodiments, the first machine learning classifier, the second machine learning classifier, or a combination thereof, is stored on a computer system separate from the camera-enabled electronic device. In some embodiments, the computer system comprises a cloud-based computer system. In some embodiments, the first machine learning classifier is configured to classify the subject's physiological state or condition of the ear as abnormal, normal, or wax. In some embodiments, the second machine learning classifier is configured to classify the subject's physiological state or condition of the ear as abnormal, myringosclerosis, otitis externa, perforation, retraction, or trauma.

Aspects of the present disclosure describe a method for identifying a physiological state or condition of an ear of a subject, comprising: processing a plurality of images or one or more video segments and/or clips from the ear of the subject to determine a time series of classification values; applying a window operation to the time series of classification values; and identifying the physiological state or condition of the ear of the subject from the windowed time series classification values.

In some embodiments, the processing comprises using a trained machine learning (ML) algorithm to process the plurality of images, the one or more video segments and/or clips, or a combination thereof. In some embodiments, the trained ML algorithm is stored on a camera-enabled electronic device. In some embodiments, the trained ML algorithm is stored on a computer system separate from the camera-enabled electronic device. In some embodiments, the computer system comprises a cloud-based computer system. In some embodiments, the window operation comprises a smoothening operation. In some embodiments, the physiological state or condition of the ear of the subject is predicted from the windowed time series of classification values.

### BRIEF DESCRIPTION OF THE FIGURES

Certain embodiments of the present disclosure will now be described by way of example only, in relation to the Figures.

FIG. 1 illustrates an otoscope system for digital otoscopy imaging, in the form of an otoscope attachment for a smartphone, as described in some embodiments herein.

FIG. 2 illustrates an image analysis system in a schematic overview, as described in some embodiments herein.

FIG. 3 illustrates an interface of an exemplary mobile otoscopy app, as described in some embodiments herein.

FIG. 4 illustrates a review application for reviewing media acquired by the otoscopy app, as described in some embodiments herein.

FIG. 5 illustrates a video playback interface of the review application, as described in some embodiments herein.

FIGS. 6A-6C illustrate methods for classification of images and video, as described in some embodiments herein.

FIG. 7 illustrates a model training workflow for an image classifier, as described in some embodiments herein.

FIG. 8 illustrates the architecture of a classification model, as described in some embodiments herein.

FIG. 9 illustrates method for training a classification model, as described in some embodiments herein.

FIGS. 10A-10F illustrate results of evaluating classification models, as described in some embodiments herein.

FIG. 11 illustrates processing devices for implementing described techniques, as described in some embodiments herein.

FIG. 12 illustrates an exemplary computer system that is programmed or otherwise configured to implement computer-implemented methods, as described in some embodiments herein.

FIG. 13 illustrates an example video play back interface with one or more tags on one or more images, one or more frames, and/or one or more clips and/or segments of video, as described in some embodiments herein.

### DETAILED DESCRIPTION

Aspects of the present disclosure, in some embodiments, provide a system for obtaining images of a subject's ear canal using a mobile device (e.g., a smartphone or other mobile or portable computing and/or communications device) with an otoscope attachment and analyzing the images to detect certain conditions.

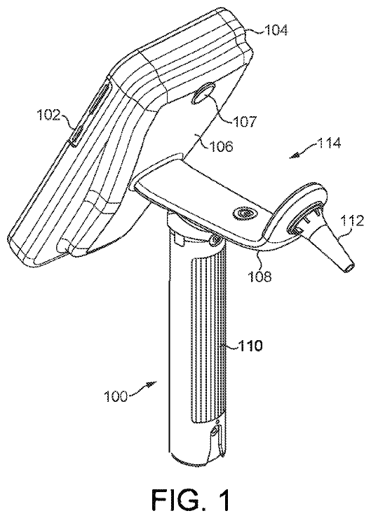

FIG. 1 illustrates an exemplary otoscope for use in the described system. The otoscope may be provided in the form of an otoscope attachment 100 for a mobile user device such as a smartphone 102 to allow use of the camera and processing capabilities of the smartphone. The otoscope attachment may comprise a housing 104 into which the mobile device can be inserted. A rear portion 106 of the housing may include an optical subsystem including an optical element (e.g., lens) 107 for projecting an image onto a camera lens and sensor of the mobile device. The rear of the housing, along with a handle 110 and a speculum 112 may be coupled to a spacing element 108. The spacing element may be arranged to mount the speculum in a fixed position relative to the optical element 107 and mobile user device 102. The speculum 112 may define an aperture through which human or animal anatomical structures, such as a subject's ear canal, may be examined. The speculum may be disposed on the distal end of the spacing element 108 and is placed in a subject's ear canal during operation of the otoscope. This may provide a fixed separation between the object being imaged (ear canal) and the optical element 107 in the housing 104. The depicted arrangement may provide a gap 114 between the speculum and the housing for tool access to the ear canal through the speculum 112, e.g., for medical procedures such as micro-suction.

The optical subsystem in the housing may include additional optical elements, such as lenses and/or mirrors, for projecting the image of the ear canal onto the mobile device camera sensor. The otoscope attachment may also include a light source (e.g., one or more LED lights) arranged, for example, in the speculum or housing to provide illumination of the inner ear. The otoscope attachment and/or one or more features of its optical subsystem may be as described in GB 2586289, U.S. Pat. No. 10,898,069, GB2569325, U.S. Pat. No. 11,696,680, and/or US2022/0409020, the entire contents of which are hereby incorporated by reference.

However, while described in relation to a particular otoscope attachment used with a smartphone or similar device, the described techniques can be used with other forms of otoscope and/or camera systems. Depending on the lighting and/or image capture capabilities of the mobile device, and its camera(s) in particular, the system may be used with a mobile device without any dedicated otoscope attachment (e.g., using a suitable macro mode and/or macro lens).

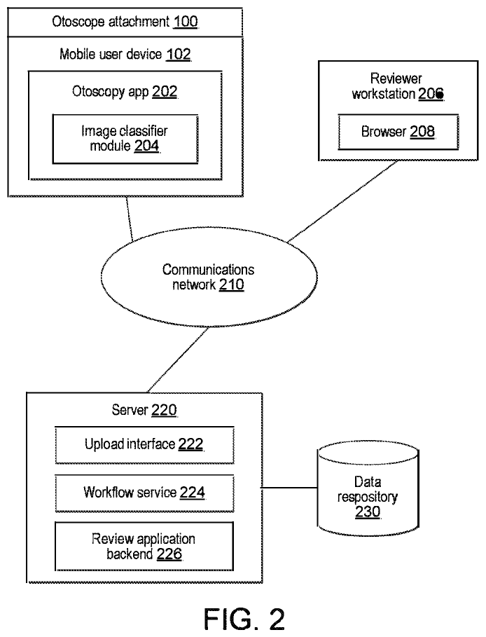

FIG. 2 illustrates an AI-supported system for otoscopic image analysis in overview. The system may comprise a mobile user device 102, which may be used to obtain one or more images and/or one or more segments or clips of video of a subject's ear canal by employing the otoscope attachment 100 as shown in FIG. 1. One or more Images and/or video clips may be obtained during a subject's appointment using an otoscopy app 202 that interfaces with the mobile device's camera subsystem to record one or more images and/or one or more video segments or clips. One or more images or one or more video clip or segment data (e.g., individual video frames, a plurality of video frames, and/or groups of frames) may be processed by an image classifier module 204, described elsewhere herein. The classifier module may provide a classification output indicating whether a given image or video frame is likely to depict an abnormal condition (e.g., disease condition) or whether it appears normal (e.g., healthy).

The otoscopy app at the mobile device may communicate with a server 220 over a communications network 210 (e.g., the Internet, intranet, and/or cellular communications network). Media (e.g., one or more images and/or one or more video clips or segments), together with classification output(s) of the classifier module generated for the media, may be uploaded by the otoscopy app to a server via an upload interface 222 provided at the server. In some embodiments, prior to uploading, the media may be filtered of at least some sensitive data of the subject such as personal information, health information, medical information, personal identification information, and the like. The server may implement a workflow service 224 which manages a review workflow for uploaded media and a review application backend 226 allowing reviewing users to access and review media in addition to e.g., associated classifications, captured by the mobile device, implemented as a stand-alone application and/or mobile, web, or Internet or intranet enabled application. A data repository 230 may store uploaded media items, along with other relevant information such as the classification(s) outputted by the classifier, subject data, user data, diagnostic information, workflow task statuses, or any combination thereof. In some cases, subject data and/or diagnostic information may comprise data of a questionnaire completed by the subject. In some cases, the questionnaire may comprise a plurality of questions that pertain to a subject's current ear complaint, previous ear history, or a combination thereof. In some cases, the plurality of questions may comprise at least about 10 questions or at least about 12 questions. In some cases, the plurality of questions may comprise up to about 10 questions, or up to about 12 questions. In some instances, the plurality of questions may comprise about 10 questions to about 12 questions. In some cases, the subject data and/or the diagnostic information may comprise a frequency response result from a hearing test of the subject. In some instances, the hearing test may comprise an air only hearing test. In some cases, the frequency response of the hearing test may comprise a plurality of frequency points measured, analyzed, and/or observed. In some cases, the plurality of frequency points may comprise at least about 4 frequency points or at least about 8 frequency points measured, analyzed, and/or observed. In some cases, the plurality of frequency points may comprise at most about 4 frequency points or at most about 8 frequency points. In some cases, the plurality of frequency points may comprise about 4 frequency points to about 8 frequency points. In some cases, the subject data and/or the diagnostic information may comprise: the subject's ear wax composition, gender, ethnicity, body weight, body mass index, blood pressure, blood glucose, pulse, or any combination thereof. In some cases, the subject data and/or the diagnostic information may comprise a measure, quantification, determination, identification, and/or characterization of one or more analytes of a biological sample of the subject. In some cases, the biological sample of the subject may comprise ear wax, a liquid biopsy (e.g., a blood sample), a tissue sample, a sample of cells from the ear canal, a sample of cells from the tympanic membrane, or any combination thereof. In some cases, the measure, quantification, determination, identification, and/or characterization of the one or more analytes of a biological sample may comprise measuring, quantifying, determining, and/or identifying DNA, RNA, nucleic acid molecules, nucleic acid molecule genomic aberrations (e.g., insertions deletion mutations (INDELS), single nucleotide polymorphism (SNIP), and/or copy number variation), or any combination thereof, of one or more nucleic acid molecules of the biological sample. In some cases, the measure, quantification, determination, identification, and/or characterization of the one or more analytes of a biological sample may comprise measuring, quantifying, determining, and/or identifying proteomic, transcriptomic, methylome, and/or epigenomic markers of the one or more analytes of the biological sample. The data repository may include one or more databases (e.g., relational) and/or other storage such as file storage. For example, one or more images and/or one more video clips or segments may be stored in a file repository and associated classification data and subject and/or appointment data may be stored in a relational database. In some cases, the subject data may comprise the subject's name, date of birth, clinical history, or any combination thereof. In some instances, the appointment data may comprise appoint date, appointment time, appointment type, or any combination thereof. In some cases, the appointment type may comprise a hearing test appointment, an ear wax removal and/or suction appointment, an ear canal, and/or ear tympanic membrane imaging appointment, or any combination thereof. The repository and/or databases may be stored at the server 220, on a device (e.g., a smartphone), and/or separately e.g., at a separate database or file server. In an embodiment, the server 220 and data repository 230 may be implemented using a suitable storage platform, such as a suitable cloud computing platform, such as Google Cloud Platform™ (GCP), Amazon Web Services (AWS), Microsoft Azure™, or any combination thereof, for processing and storage functions.

A reviewer may access the system using a reviewer workstation 206 (e.g., a mobile device, laptop, and/or desktop computer), by using, e.g., a stand-alone application, a mobile app and/or a web browser 208 to provide an application front-end for the review application backend 226 implemented at the server.

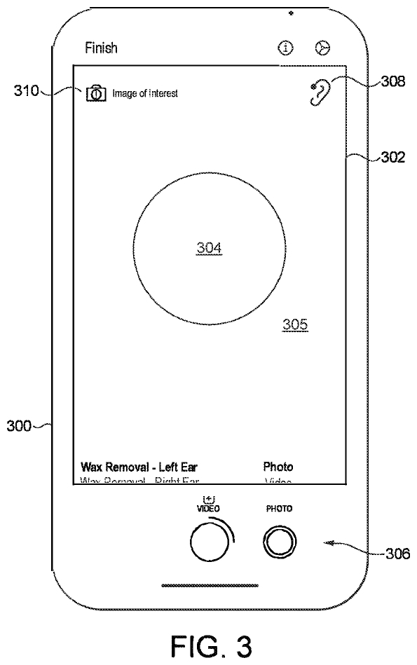

FIG. 3 illustrates by way of example an implementation of a user interface 300 for the mobile otoscopy application 202. The application can be used by an audiology practitioner or other medical professionals or paraprofessionals for otoscopy imaging and during procedures such as micro suction and/or wax removal to provide the user with a clearer view of the subject's ear canal to support the procedure.

The user interface 300 may comprise an image preview region 302 showing a current view of the image and/or video captured by the device camera and buttons 306 for starting and/or stopping recording of one or more video clips or segments and/or acquiring one or more images. The view of the ear canal obtained through the speculum may be displayed as a central circular illuminated region 304, surrounded by a background region 305. In some cases, the background region may comprise a darker color compared to the central circular illuminated region 304. An icon 308 may be displayed when the image classifier is active to indicate that the AI classifier, described elsewhere herein, is actively analyzing the one or more images and/or one or more video clips or segments for potential issues. Further, an icon and notification 310 may be displayed overlaid on an image if an acquired image or video clip has been classified by the classifier as an image of interest (e.g., being classified as abnormal).

In some embodiments, the AI classifier may be activated when video recording is started by the user when the user presses the video button and remains active until video recording ends. Icon 308 may be displayed during that time and the user can observe the video as it is captured in the live preview 302. During recording, frames of the captured video may be processed by the image classifier in real time. In some cases, real-time processing may comprise a frame rate of process of at least about 1 frames/second, at least about 5 frames/second, at least about 10 frames/second, at least about 15 frames/second, at least about 20 frames/second, at least about 25 frames/second, at least about 30 frames/second, or at least about 35 frame/second. In some cases, real-time processing may comprise a frame rate of process of at most about 1 frames/second, at most about 5 frames/second, at most about 10 frames/second, at most about 15 frames/second, at most about 20 frames/second, at most about 25 frames/second, at most about 30 frames/second, or at most about 35 frame/second. If a frame is labelled with an abnormal classification, then the “image of interest” notification 310 may be shown, immediately alerting the user to a potential issue. After video recording has ended, the “image of interest” flag may remain displayed for a period of time. In some cases, the period of time may comprise at least about 1 second, at least about 2 seconds, at least about 3 seconds, at least about 5 seconds, or at least about 10 seconds before disappearing. In some cases, the period of time may comprise up to about 1 second, up to about 2 seconds, up to about 3 seconds, up to about 5 seconds, or up to about 10 seconds before disappearing.

The user may alternatively obtain a single image using the “photo” button which may be immediately processed by the AI classifier, where the “image of interest” notification may be shown if an abnormal classification, described elsewhere herein, is found. The user may record a separate image during video capture by pressing the photo button. When a photo is taken there may be a noticeable flash and/or brightening on the screen and if the AI has detected an abnormality, a display of the image of interest flag may be provided. The flag may remain visible for a short period while the interface resumes live view and/or video recording to alert the user that the AI has identified a potential abnormality, health condition, or a combination thereof.

The application 202 may support separate otoscopy and micro suction and/or wax removal modes in which case the AI classifier may be activated only during otoscopy, not in the micro suction mode since the image will typically be obscured by micro suction tools.

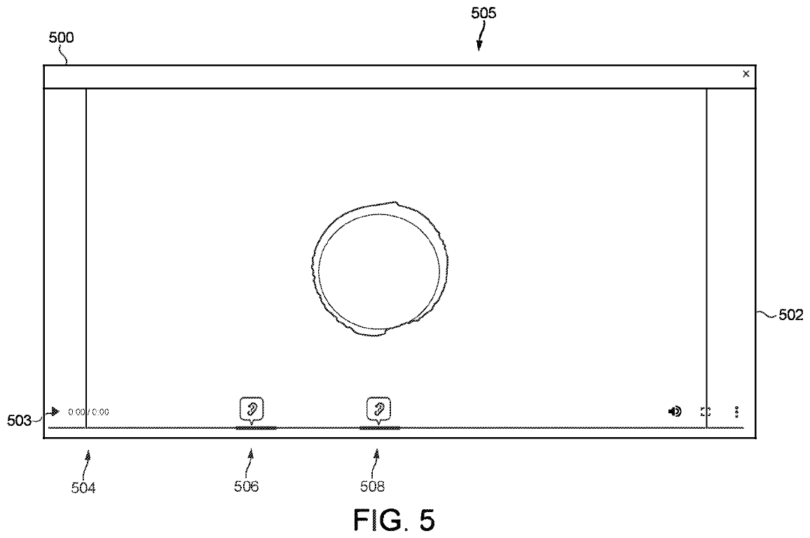

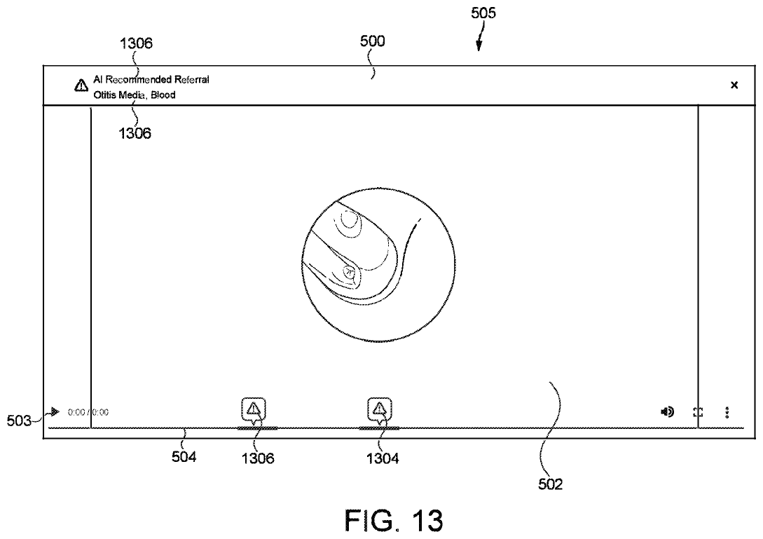

Images and videos classified as abnormal by the classifier may be tagged with one or more tags (506, 508, 1304, 1306) with information based on the classification, including a tag to indicate that the one or more image(s) and/or one or more video segments and/or clip(s) should be reviewed and/or the proposed diagnosis classification, as shown in FIGS. 5 and 13. In some cases, the tags may comprise the subject data and/or diagnostic information, described elsewhere herein. The tags may be stored with the one or more image(s) and/or one or more video segments and/or clip(s) in a media library. The application may also provide a gallery view showing media (images and videos) that have been obtained; any tagged as potentially abnormal and/or requiring review are moved to the top of the list in the interface. In certain implementations, the interface may allow the user to modify the review recommendation and/or classification(s) output by the classifier in which case the stored media tags may be modified accordingly.

For videos, classifier tags may specify the location(s) in the one or more video segments and/or clips, e.g., the specific one or more frames (or frame ranges), where an abnormality was classified. Such a location may be specified, for example, as a time or frame index. Thus, one or more video segment and/or clips may have multiple classifier tags associated with it, corresponding to different locations where one or more frames and/or one or more images were tagged as abnormal. In some cases, classifier tags may specific a location or portion of one or more frames (e.g., a region of one or more pixels of a frame) that correspond with a classification (e.g., abnormal, normal, and/or wax, as described elsewhere herein).

References to “tags” or “tagging” may indicate that some information (e.g., classifications) is stored that is associated with a media item such as an image, video clip or video frame. The tag information may be stored as part of the media item (e.g., in metadata within a media file) or as a separate data structure linked to the media item (e.g., by a file path and/or name and where applicable, frame identifier).

After acquisition by the application (and, if appropriate, local review by the practitioner on the mobile device), relevant images and video clips may be uploaded to the server, either automatically and/or on request by the user. In one approach, only those images and videos tagged requiring review and/or assigned an abnormal classification may be uploaded. Alternatively, all media items may be uploaded. The uploaded information may comprise the classification tags, i.e., the classifications assigned to images, frames, and/or videos. For video clips, the locations in the video of any frames classified as abnormal may also be specified in the classification data uploaded with the media items. In some cases, images and video clips may be grouped by appointment, and/or appointment data (e.g., subject, time and/or date) associated with the media items prior to being uploaded.

Referring back to FIG. 2, in some embodiments, the server may store appointment information comprising the images and/or video clips tagged for review with the associated classification data and any associated appointment, subject data, and/or diagnostic information, described elsewhere herein, in the data repository 230. If media items have been tagged for review, then the workflow service 224 may add the appointment (or one or more media items) to a workflow queue to indicate that review is required. Appointments may be assigned to specific reviewers or may be made available to a pool of reviewers.

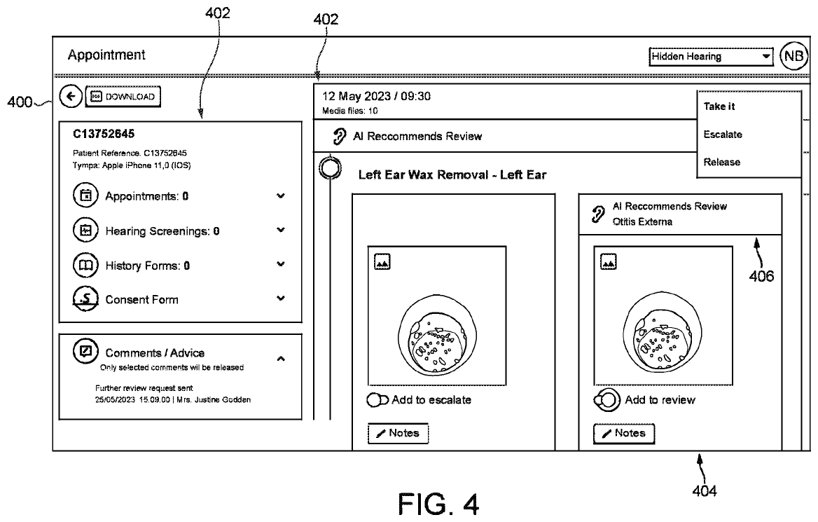

A reviewer may access the review application 226 using reviewer workstation 206. A list of appointments for review may displayed (which may be specifically assigned to the reviewer, or a general work queue) and the reviewer may select an assignment to review. An example user interface 400 of the review application (implemented as a web application) is shown in FIG. 4. The interface may display appointment information 402 (e.g., subject name or reference, time and/or date, etc.) and a list and/or gallery view 404 showing media items. For example, the media items may comprise all media recorded and/or media items flagged for review by the AI classifier tagged with the AI review recommendation and suggested diagnostic classification (406). Alternatively or in combination with the suggested diagnostic classification, the media items may comprise a recommendation for action. Interface controls may provide for adding and/or removing items to and/or from the review and for marking items for escalation. The user can select an item to display a larger view of an image or to play back a video clip to allow the reviewer to assess the media and determine whether they agree with the AI classification. If necessary, the reviewer can override the AI classification, for example by specifying an alternative diagnostic classification and/or marking the item as normal (e.g., healthy) and/or not requiring review. If necessary, the reviewer can override the AI classification, for example by specifying an alternative recommendation for action and/or marking the item as normal (e.g., healthy) and/or not requiring review. Any modified classifications assigned by the reviewer following review may then be stored with (and/or linked to) the media item in the repository.

FIGS. 5 and 13 show a video playback interface 505 displayed when the reviewer has selected a video clip to review. The interface may comprise a video region 502, playback controls 503, a timeline and/or progress bar 504 showing a current playback location and allowing the user to skip to a particular place in the video, a video header 500, or any combination thereof. Any frames and/or regions tagged by the AI as abnormal or requiring review may be labelled on the timeline with markers (e.g., 506, 508, 1304, 1306) and a relevant section of the timeline containing those frame(s) may be highlighted. In some cases, the video header 500 may comprise information and/or a text object 1306 pertaining to the one or more tags of the one or more frames and/or one or more video segments and/or clips, as shown in FIG. 13. In some cases, the information and/or text object 1306 may indicate a classification, determination, and/or diagnosis, as described elsewhere herein, e.g., a classification of Otitis Media and/or blood in one or more images, one or more frames, one or more video segments and/or clips, or any combination thereof. In some cases, the one or more tags (506, 508, 1304, 1306) may comprise a visual object e.g., a graphic object indicating a machine learning algorithm and/or predictive model classification and/or alert, and/or a filled and/or colored region of the progress bar 504 corresponding to the tagged one or more images, one or more frames, one or more video segments and/or clips, or any combination thereof, as shown in FIGS. 5 and 13. In some cases, the timeline marker labels may use the location information previously associated with the video clip by the classifier at the mobile device when detecting the abnormal frames. The reviewer can click on a marker (and/or directly on the timeline) to skip to the tagged location, i.e., to the region containing the frame(s) for which the classifier outputted an abnormal classification. To allow review in context, selection of the marker may trigger playback from a time index slightly earlier than the tagged frame, for example, a time index preceding the tagged frame by a given lead-in time—e.g., up to about 10 seconds earlier than the tagged frame.

This approach may allow the reviewer to find the relevant parts of the video efficiently and thus speeds up the clinical review and decision-making process. The diagnostic classification assigned to the video may be displayed either for the whole video or for individual segments; for example, during playback, an overlay may be displayed during a segment of the video that includes the tagged frame(s), with the overlay showing the AI classification generated for the frames. In some cases, the diagnostic classification assigned to one or more frames may be displayed either for the whole image of a given frame and/or for individual portions and/or segments of the image. In some cases, alternatively or in combination with the diagnostic classification, the recommendation indication or recommendation for action assigned to one or more frames may be displayed either for the whole image of a given frame and/or for individual portions and/or segments of the image.

### AI Classification Model

The AI classifier 204 may use a machine learning model to interpret otoscopic images, supporting end users making more consistent referral decisions. The AI system may use models to predict classification classes, given images of subject's ear canals.

The prediction process may be split into two stages. The first stage, Stage 1, may determine whether an image is ABNORMAL, NORMAL, or WAX. shows these exemplary classes.

TABLE 1
Example Stage 1 Classes
Classes

ABNORMAL
NORMAL
WAX

This initial classification may provide a high level, first classification of the image. NORMAL and WAX can be treated as “normal” classifications (which typically do not require further investigation) but are provided as separate classifications due to the value in knowing that earwax is present, such as indicating to the user that earwax removal may be helpful.

If the prediction is ABNORMAL, then such image(s) and/or video clips may be considered as a referral case. These cases may then be passed onto the next stage, where a diagnosis is predicted. Splitting the classification into two stages may reduce the pressure on a single model learning all these classes. Similar looking classes can introduce confusion within one model, among other issues that are alleviated by using separate models.

The next stage, Stage 2, may be used to determine a specific diagnostic sub-class of ABNORMAL that the ABNORMAL predictions fall into. shows an example of a set of classes used in an implementation, covering conditions that might be diagnosed based on otoscopic imaging.

TABLE 2
Example Stage 2 Classes
Classes

ABNORMAL
MYRINGOSCLEROSIS
OTITIS_EXTERNA
PERFORATION
RETRACTION
TRAUMA

ABNORMAL here may indicate a generic abnormal class. This may cover cases where the training data, described elsewhere herein, is not sufficient to learn a separate class to an acceptable level. However, providing this generic class may allow the model to indicate that an image was not in any of the other classes known by the model. The remaining classes may be selected based on the fact that the training data may comprise enough examples images and/or video training data for the model to develop an accurate prediction of the classification of these classes. The abnormal classes may comprise ear conditions seen in the population. Thus, application of both models may yield a combined Stage 1 and stage 2 classification e.g., ABNORMAL, and TRAUMA or ABNORMAL, and ABNORMAL.

The specific classifications are given by way of example. In practice, the classes may be adapted based on the specific requirements of the implementation and the available training data.

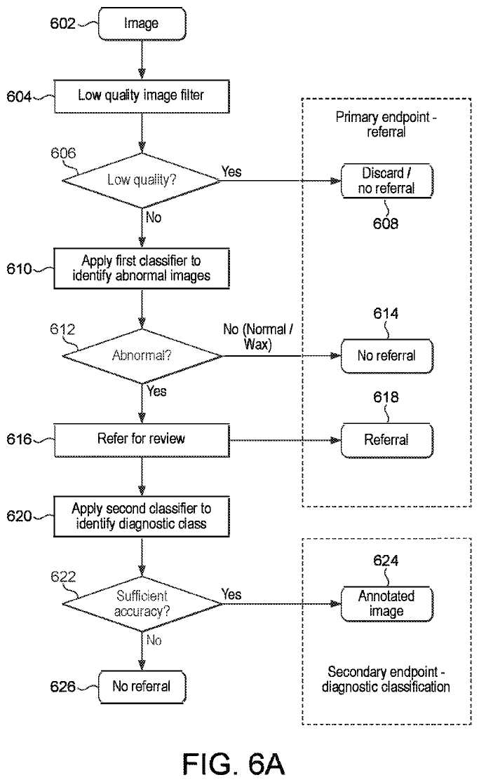

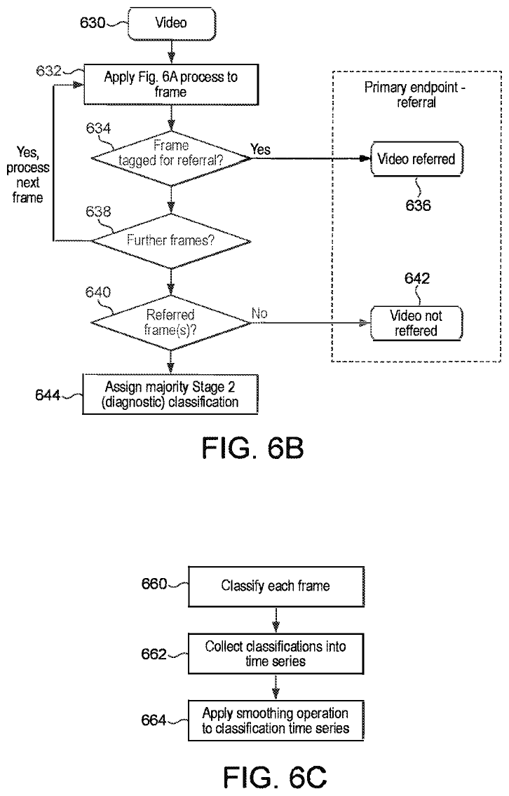

FIG. 6A shows a classification workflow. Both Stage 1 and Stage 2 classification are shown in this flowchart, illustrating how images may be predicted by Stage 1 as needing referral that are then passed to Stage 2. In some cases, both stage 1 and/or stage 2 classification machine learning algorithms, machine learning models, and/or predictive models may provide an output classification to as an input into a third machine learning algorithm, machine learning model, and/or predictive model that may output a classification (e.g., normal, abnormal, wax, myringosclerosis, otitis externa, perforation, retraction, trauma, or any combination thereof classifications of the one or more images, one or more frames of one or more videos, one or more videos, one or more video segments and/or clips, or any combination thereof. In some cases, the classification workflow may comprise a combination of a plurality of stages in series and/or in parallel.

Specifically, the process may start by processing an input image 602 (e.g., one or more images or a frame from a video clip) by a low-quality filter 604 to filter out low quality images (that might not be classified accurately by the models). In some cases, the filter may comprise a machine learning algorithm, machine learning model, and/or predictive model, e.g., a convolutional neural network, which outputs a value from 0 to 1.0 when provided an input one or more frames, where the value indicates the quality of the image. In some cases, a value of 1 outputted by the machine learning model, machine learning algorithm, and/or predictive model may indicate a suitable quality for further processing and/or classification. In some cases, the filter may analyze one or more metrics of one or more images, one or more frames, one or more video clips, any portions thereof, or any combination thereof. In some cases, the one or more metrics may comprise image variance, image intensity, image color, or any combination thereof. In an implementation, this step may be performed using an image pre-processor that may utilize a dedicated machine learning model, e.g., a binary classifier classifying images as adequate or inadequate quality for use in the ML pipeline. Alternatively, this step can be performed by the practitioner (e.g., Ear Nose and Throat specialist) for images and/or video clips based on manual review. If the input image is considered of low quality (test 606) it may be tagged as “no referral”, meaning that it will not be processed further or passed for review and may be discarded.

If the image passes the quality filter 604, the Stage 1 classifier model may be applied to the image in step 610 to generate a normal, wax, or abnormal classification. Test 612 may determine whether the image requires referral. In some cases, images classified as “normal” or “wax” may be determined to require no referral (output 614) and the process ends.

If the image is classified by the Stage 1 classifier as “abnormal” then the image may be tagged for referral in step 616 (referral decision output 618). The image may then be further processed by the Stage 2 classifier in step 620 to determine a diagnostic classification (as per example classifications shown in Table 2). Alternatively or in combination to the diagnostic classification, the image may be further processed to determine a recommendation indication or recommendation for action.

In some embodiments, in addition to the classification itself, the Stage 2 classifier model may output a prediction probability indicating an estimated accuracy and/or confidence relating to the classification. In some cases, the accuracy and/or confidence may comprise and accuracy and/or confidence of at least about 80%, at least about 85%, at least about 90%, at least about 92%, at least about 95%, or at least about 98%. In some cases, the accuracy and/or confidence may comprise and accuracy and/or confidence of at most about 80%, at most about 85%, at most about 90%, at most about 92%, at most about 95%, or at most about 98%. This may be compared to a threshold (622). If the classification meets the accuracy threshold, the image may be annotated (e.g., tagged) with the diagnostic classification generated by the Stage 2 classifier (output 624) and/or the recommendation indication or recommendation for action. If, on the other hand, the classification accuracy is low (below the threshold), then the output may be “no referral” (626) (which may override the previous referral decision at 618) or “no recommendation.” In some cases, the threshold may comprise at least about 75%, at least about 80%, at least about 85%, at least about 90%, or at least about 95%. In some cases, the threshold may comprise up to about 75%, up to about 80%, up to about 85%, up to about 90%, or up to about 95%.

For video, the workflow may be similar to that for images described elsewhere herein, except that a video can be interpreted as a collection of frames. In some embodiments, the process may be illustrated in FIG. 6B and involves running the FIG. 6A process (step 632) for each frame of the input video 630 by looping steps 632-638. If an abnormal frame is detected by the model and the frame is thus tagged to be referred for review (634) by the Stage 1 classifier, then the video may be considered abnormal, and may be tagged for referral (636). If no frames are tagged for referral (640) then the video may not be tagged for referral (642).

For the Stage 2 classification, it is possible that classifications of different frames in a video may produce inconsistent results (e.g., producing different diagnostic classes and/or different recommendation indications or recommendations for action). To address this, the system may select an overall classification for the video (step 644) based on the winning class under a criteria (e.g., a majority, and/or consensus class). In some cases, a consensus may comprise an agreement of a classification of a plurality of health care personnel (e.g., attending ear nose and throat physicians) for one or more images, one or more frames, one or more video segments and/or clips, or any combination thereof. In some cases, the consensus may comprise an agreement of a classification between two or more health care personnel for one or more images, one or more frames, one or more video segments and/or clips, or any combination thereof. In some cases, a minimum class probability (for example at least about 0.75) may be required for a frame classification to be considered as accurate enough. Then, the most frequently predicted class across the frames of the video with probability meeting the threshold may be assigned to the video. In some cases, one or more predicted class may be assigned to a video, where a first classification may be assigned to a first portion, segment and/or clip of a video and a second classification may be assigned to a second portion, segment, and/or clip of the video, where the first portion, segment and/or clip differ from the second portion, segment, and/or clip of the video. In some cases, the first classification may comprise a wax classification and the second classification may comprise an abnormal classification.

In certain embodiments, the classification methodology for videos may be further refined by way of additional post-processing as illustrated in FIG. 6C. Firstly, Stage 1 and (where applicable) Stage 2 classifications may be obtained for each video frame using the FIG. 6A process (step 660), by applying the Stage 1 and Stage 2 classifiers to the video frames.

These classifications may then be collected (step 662) as a time series, forming a sequence of classifications over time. A smoothing operation may then be applied to the time series of classifications (step 664). This is accomplished through the application of a median window function with a window size, in this example, a window size of up to about three frames (though other window sizes could be used). In some cases, the window size may comprise at least about 2 frames, at least about 3 frames, at least about 4 frames, at least about 5 frames, at least bout 6 frames, at least about 7 frames, at least about 8 frames, at least about 9 frames, at least about 10 frames, at least about 11 frames, at least about 12 frames, at least about 13 frames, at least about 14 frames, at least about 15 frames, at least about 16 frames, or at least about 17 frames. In some cases, the window size may comprise up to about 2 frames, up to about 3 frames, up to about 4 frames, up to about 5 frames, up to about 6 frames, up to about 7 frames, up to about 8 frames, up to about 9 frames, up to about 10 frames, up to about 11 frames, up to about 12 frames, up to about 13 frames, up to 14 frames, up to about 15 frames, up to about 16 frames, or up to about 17 frames. In some cases, the window size may comprise about 3 frames to about 15 frames. The median prediction value may be computed within each window, meaning that for every three consecutive frames, the median prediction from the model may be taken as the representative value for that segment of the video. Additionally, a model probability threshold (e.g., at least about 0.75), as described elsewhere herein, may be considered as the minimum threshold for the frame to be taken into account in the sliding window.

The utilization of a median window function for smoothing the time series may aid in reducing the impact of potential noise and/or outliers in the predictions, contributing to more stable and accurate results. In some cases, a mean and/or mode form of averaging or summarizing function may be used. In some cases, the window function may be applied as frames are classified rather than at the end once all frames have been classified.

After the application of the median window function a video may be tagged for referral if it contains a frame that was marked as abnormal (e.g., by the Stage 1 classifier). If video does not contain any abnormal frames the video may not be marked for referral. For videos tagged as abnormal, a majority Stage 2 classification may then be derived as described elsewhere herein.

In some cases, the process of video classification using an image-trained model may involve the generation of predictions on one or more frames in addition to the application of post-processing techniques to handle time series data. By employing a median window function for smoothing and histogram analysis for decision-making, this approach can ensure accurate, reliable, and meaningful video classifications.

Different implementations may apply temporal smoothing to either, both, or neither of the Stage 1 and/or Stage 2 classifications, described elsewhere herein. In some cases, the histogram analysis may comprise a histogram of a mean, median, standard deviation, skewness, kurtosis, bimodality index, or any combination thereof, of a plurality of pixels of one or more images, one or more frames, one or more video segments and/or clips, or any combination thereof.

In some implementations, an entire subject's case (e.g., imaging conducted during an appointment) may be assigned a referral decision based on the classifications of any images and/or videos that were captured as part of the appointment. In that case, for example, the appointment may be referred where any image or video was tagged for referral.

In some embodiments, both Stage 1 and/or Stage 2 classifiers may be implemented as artificial neural networks (ANNs). Design of the neural networks is described elsewhere herein. However, other types of machine learning models as described herein could be used for either or both stages.

As noted above, the Stage 1 and/or Stage 2 classifications and referral decision may be stored as classification data linked to the relevant media items. For video clips, the classification data may link abnormal classifications to specific locations within the video where frames were classified as abnormal (e.g., via a frame or time index). At some point, for example, on user request, or automatically on completion of an appointment, acquired media items with their associated classification data as generated by the FIG. 6A-6C processes may be uploaded to the server for review.

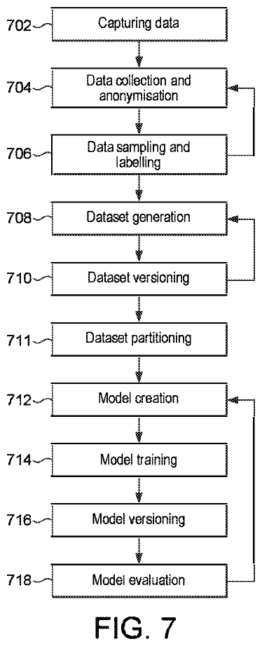

In some embodiments, FIG. 7 illustrates an example model training process for the AI classifier models. The description assumes a data repository and processing functions implemented on a computing platform such as a cloud-based platform, e.g., Google Cloud Platform™ (GCP). In some cases, model training may be conducted and/or implemented on a device, e.g., a smartphone device, as described elsewhere herein.

In some embodiments, the process may start (step 702) with capturing source data for training the models. Inputs to this step may comprise a set of images taken e.g., using an otoscopy system as described with reference to FIG. 1. These images may be collected during a subject's appointment and are stored in a database at the server together with associated subject and/or appointment data.

In step 704, data collection and anonymization may be performed. This may involve anonymizing data associated with each image and the anonymized data, along with the images, may be moved to a Machine Learning (ML) environment in the repository. For example, images may be copied to a memory bucket (e.g., a Google Cloud Platform™ memory bucket) within the Machine Learning environment, with their location file path having also being anonymized.

In step 706, images may be prepared for labelling by reviewers. In one implementation, labelling “projects” may be setup on a labelling interface as shown in FIG. 4 (e.g., Labelbox™), where reviewers can provide labels for the images. In one approach, two independent labelers may submit a label, while a third labeler approves or rejects the labels. Labelers may be experienced ear, nose, and throat specialists and/or audiologists. The labels may be associated with the images and stored in the repository.

Data collection and anonymization may be an on-going process that may be applied to new data as it is received at the server. However, the data sampling and labelling stage may be applied on a batch basis instead of continuously, whereby periodically new data is sent for labelling. In some cases, a batch of images for labelling may comprise at least about 3 images, at least about 5 images, at least about 10 images, at least about 15 images, or at least about 20 images. In some cases, a batch of images for labelling may comprise up to about 3 images, up to about 5 images, up to about 10 images, up to about 15 images, up to about 20 images. In some cases, a batch of images for labelling may comprise about 3 images to about 20 images. In some cases, batching a plurality of images may simplify, facilitate, and/or streamline a labelling process for health care personnel, e.g., ear nose and throat physicians. Also, based on the number of images for each classification in the labelled data, the data collection process can be tailored to focus more on certain types of images.

The system may run the low-quality image filter, described elsewhere herein, at step 702 or 704 to identify low quality images which can then be discarded to avoid having poor quality images labelled and included in the training set.

The dataset generation stage 708 may use the anonymized data recorded for images and the labels assigned by the reviewers to create training datasets, for example in the form of comma separated value (CSV) datasets using the reviewer-assigned labels, which are linked to the image locations of the associated images in the data repository.

Dataset versioning stage 710 may involve assigning a version to a dataset whereby its contents may be recorded in a relational database in the Machine Learning environment. The datasets may be uploaded to cloud platform (e.g., GCP) locations in the Machine Learning environment that reflect the version assigned to the dataset.

Steps 708-710 may be repeated to create multiple datasets from the same starting set of images and labels. In some cases, different inclusion criteria can be used to generate the final dataset (e.g., CSVs). In some cases, inclusion criteria may comprise one or more pathologies of a subject e.g., myringosclerosis, otitis externa, perforation, retraction, and/or trauma, described elsewhere herein; hearing loss characterization; demographic information of the subject; the number of images in a dataset; or any combination thereof. In some cases, demographic information may comprise a subject's age, gender, socioeconomic status, geographic location of residency, body mass index, ethnicity, ethnic background, or any combination thereof.

For training purposes, the dataset may be split in step 711 into three partitions by subject identifier: a training partition, a validation partition, and a test partition.

Model creation step 712 may initialize a model in accordance with a provided set of hyperparameters for the model's architecture (e.g., layer numbers and/or sizes etc.) This may create a model object in the ML environment ready to be trained.

The model training step 714 may then be invoked using training parameters, including the dataset version to be used for training. The newly created model may be trained on the training partition of the specified dataset version. This dataset may define the mapping between images (e.g., in terms of their memory locations and/or file paths) and their assigned classification labels. This step may produce a trained model as output.

In step 716, a version may be assigned to the model based on the dataset version. This version may be used to determine where evaluation results and the model itself are stored in the repository.

In some cases, Step 718 may receive the newly trained model, dataset version and/or model version as input. The provided dataset version may be used to load a validation split of the dataset. The model's performance may be evaluated by applying the model to the unseen data in the validation split. Evaluation metrics may be calculated based on the model's performance on this validation data. The results and the trained model may be stored in the data repository in a location specified via the model version.

Training and evaluation of models can be repeated, with multiple models trained on the same dataset, for example to tune the training parameters for the model. The results of the model evaluation can be used to improve the training of the next generation of models.

While described in relation to images, the same training pipeline, described elsewhere herein, may be applied to videos, by splitting videos into one or more frames and training the models on the one or more frames.

The above process may be performed at the server (220). Once a model has been trained to achieve acceptable performance, the model may be pushed to the mobile device(s) for use by the mobile otoscopy application at the device(s) in performing local on-device classification. While in the described implementation classification is performed at the mobile device, the model may also be used at the server for classification (or re-classification), e.g., as part of the review workflow/application.

### Capturing Data (702)

During appointments, images of subject's ear canals and/or eardrums may be taken using the otoscopy system. Any suitable image formats and resolutions may be used; in an example implementation, images have the following properties: Resolution: 3024×4032 (iOS), 3456×4608 (Android); Color format: RGB; Compression: JPEG and/or PNG; Pixel Intensity Value Range: [0,255]; or any combination thereof. On average, the region of interest (ROI, the specific area or part of an image that contains the relevant content and must be interpreted for labelling) in these images may be around 1600×1600 pixels.

The images may be associated with a subject and appointment, where such data of the subject and appointment data is available to later stages in the workflow, described elsewhere herein. The image may be stored in a location in the production storage buckets on cloud platform. The data relating to the image, along with this cloud memory location, may be written to a production SQL database.

### Data Collection and Data Anonymisation (704)

In some cases, the data collection and/or data anonymisation 704 stage may move data into the Machine Learning environment, consisting of cloud memory buckets for the media and a SQL database for information relating to the media. This SQL database may be referred to as, e.g., the machine learning operations (MLOps) database. Once the data is in this environment, is the data may be anonymized and separated from the original clinical data.

The anonymisation step may anonymizes any subject identifiable data. Examples of data that may be anonymized for a record may include a media identifier, subject identifier, media date and/or time of upload, media path (file name and/or path to storage), video path (file name and/or path to storage), label identifier, label date and/or time, appointment identifier, appointment date, or any combination thereof. A hash function such as BLAKE2b may be used to anonymize values such as identifiers whilst date scrambling may be used for date fields. The anonymized data may then be moved to the cloud based (e.g., Google Virtual Platform (GVP)) storage for the ML environment, and data relating to the image including the new cloud memory location is written to the MLOps database.

### Data Sampling and Labelling (706)

In some cases, a labelling interface (e.g., Labelbox™, available from Labelbox, Inc. of San Francisco, CA may be used to generate and store image annotations and/or labels. The data used on the labelling interface may be sourced from the MLOps database. In some cases, media may not be uploaded and may instead be referenced by the labelling interface. Media IDs (anonymized) may be attached such that the labels can be exported later and linked to media. Media may be organized into labelling projects and labelers and reviewers can then add annotations including classification labels for the media.

### Dataset Generation (708)

Dataset generation may comprise the process of exporting the labels stored within the labelling interface and creating a dataset (e.g., a CSV dataset) of media paths and/or labels. Fields included in the CSV datasets in an example implementation may comprise: Media ID, Label and Media File Path as string values, or a combination thereof.

First, the labels assigned by reviewers may be exported from a set of the labelling interface (e.g., Labelbox™) projects and are processed such that only labels inferred to have the “Done” status are kept. In some cases, if the label is from a consensus project, only the winning annotation may be kept. Labels may then be mapped to the desired set of model classes or excluded if outside this set. The media ID associated with each label may then be used to map each label to a media path.

Metadata for the dataset may then calculated. In some cases, metadata may comprise: split sizes, class distributions, classes used, or any combination thereof. The dataset may be assigned a version and relevant information may be recorded in the MLOps database. The metadata and dataset (e.g., CSVs) may then be uploaded to a location on the cloud platform, based on the version assigned to the dataset.

### Dataset Versioning (710)

The version of the model can be tracked in many ways and formats. In some cases, model versions may be defined in the form: vX.YYWW.N, where: X may comprise a value decided by the creator of the dataset, e.g., 0; YY may comprise the last 2 digits of the current year, e.g., 2023 would be represented by 23; WW may comprise the current week of this week, e.g., the first week of the year would be 01; and N may comprise the next version number available for this particular version. For example, if v0.2319.1 and v0.2319.2 already exist, N may be 3. The version number may be worked out by querying the MLOps database for existing dataset versions.

### Image Processing

Images may be pre-processed prior to model training, including to crop the images. Pre-processing may occur, for example, as part of the dataset generation step, or as a separate step prior to model training.

Cropping may comprise identifying and/or finding the region of interest (ROI) in an image. In some cases, the ROI may comprise the illuminated central region (as shown in FIG. 3, region 304) where the ear canal is visible. The ROI may be found e.g., by using standard image segmentation techniques e.g., binary thresholding, adaptive thresholding (e.g., Nobuyuki Otsu (OTSU) image thresholding). The ROI may be defined as a square region (whereas the raw image need not be square) and extended by some small factor (e.g., to a size of 1.3*ROI). The highest possible resolution may be maintained in the final image to maintain quality. The resulting images may thus have varied (e.g., square) resolution.

The resulting images may be referred to as pre-cropped images, as described elsewhere herein. In some cases, the pre-copped e raw images may be cropped and stored in a separate location of cloud memory. The location on cloud memory to load images from can be defined during the loading of a dataset. With this structure, the type of images used (raw and/or cropped) can be decided at training time.

Further pre-processing may be performed when the pre-cropped images are loaded into memory during the model training pipeline.

Prior to model training, the images may be read from the cloud memory repository and decoded into an object (e.g., an array representation) in memory. A scaling operation may convert each image to a float image representation with pixel intensities in the range [0.0,1.0] by multiplying the pixel values by 1/255. This scaling can reduce the impact of the vanishing gradient problem since larger inputs to a neural network can cause difficulties during the learning process.

In some cases, ahead of resizing the one or more images, described elsewhere herein, the images may comprise a size of about 4,000 pixels by about 4,000 pixels. In some cases, a foreground area and/or active area of the one or more images may comprise a size of about 900 pixels to about 900 pixels. The images may then be resized to the desired size used for training (e.g., images may be resized to 224×224 pixels in size), using bi-linear interpolation. Because the output image may be square, to avoid distortions, the aspect ratio of the image being resized may also be a square (which is ensured by the cropping step described elsewhere herein). The final size can be adapted to the requirements of the implementation. For example, the example size, described elsewhere herein, was set to be reduce the slow down training for larger models thus streamlining and facilitating the training of the large model. Furthermore, image size of 224×224 pixels may be an image size used with a neural network implementation when using transfer learning.

In an augmentation step, image transformations may be randomly applied to images when they are used during model training. In an example implementation, each image may be flipped horizontally and/or vertically in accordance with a defined flip probability. Furthermore, each image may be rotated by 90 degrees in accordance with a defined rotation probability. The use of 90-degree rotations may ensure that no interpolation is needed to implement the rotations. In some cases, an even proportion of one or more images, one or more frames, and/or one or more video segments and/or clips may be flipped horizontally, flipped vertically, and/or rotated.

### Model Creation (712)

In some embodiments, the models used for classification (Stage 1 and/or Stage 2) may comprise a neural network. In some cases, aspects of the models, described elsewhere herein, may be chosen to limit overfitting, which was found to be a challenge given the small size of the available training sets.

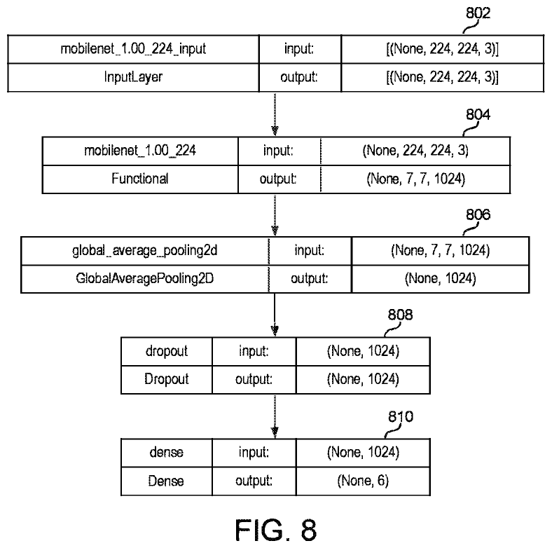

FIG. 8 shows, by way of example, a visualization of the structure of a neural network for the 6-class classifier in Stage 2. The exemplary input and output dimensions for each layer are shown in FIG. 8. In some cases, the layers of the model include: an input layer 802 (e.g., an input layer to a pre-trained MobileNet network); a MobileNet layer 804 (e.g., a convolutional component of the MobileNet network) where all of the weights in the MobileNet layer are trainable; an average pooling layer 806; a dropout layer 808; an output layer 810 comprising a dense layer with an output per class and a softmax activation, or any combination thereof. For the A Stage 2 classifier, described elsewhere herein, may comprise 6 outputs corresponding to the exemplary classifications provided in Table 2, whereas the corresponding Stage 1 classifier may comprise three output values corresponding to the exemplary three Stage 1 classifications provided in Table 1.

The “None” values that are present may indicate batch size, which indicate that the model can be provided with an arbitrary number of images during a training iteration.

In an example implementation, the same model architecture may be used for both Stage 1 and/or Stage 2 classifiers, whereby the Stage 1 and Stage 2 classifier may comprise a different number of output value.

The input layer may comprise an input dimension of 224×224×3, corresponding to a 224×224 pixel array image with three red, green, blue (RGB) color components (after pre-processing, cropping, scaling, or any combination thereof, described elsewhere herein).

MobileNet (layer 804) may comprise a pre-trained model selected for transfer learning (e.g., the model's convolutional layer and/or component). Transfer learning is a useful technique that utilizes powerful features from pre-trained networks, which are otherwise very difficult to learn on small datasets without overfitting. MobileNet may be a “small” network, which makes it more desirable for use in mobile applications. The pre-trained convolutional layer may abstract the process of extracting relevant features and map the image into a different feature space which the subsequent model layers learn from. In an example implementation, the convolutional layer may perform 3×3 depth wise convolution (e.g., for each channel separately), then 1×1 pointwise convolution across all channels (e.g., a 1×1×3 kernel). In some cases, a plurality of pointwise convolutions may be applied to the depth wise-convoluted image as required to generate any number of output channels.

In some embodiments, the convolutional layer may receive the (224, 224, 3) resolution input image and produce an output with dimensions (7, 7, 1024) (e.g., providing 1024 features at 7×7 spatial locations).

The remaining layers of the model may be referred to as the Fully Connected Component (FCC) and may be designed to provide a network that is efficient to train. While a more complex FCC may be used, a more complex FCC may stall learning or result in uncontrolled overfitting. Hence, the simpler FCC was found to work well, despite having a lower learning capacity.

The global 2D average pooling layer 806 may be used to connect the convolutional component output with the FCC input. This is because the convolutional component output may not be flat (e.g., a multi-dimensional vector). In some cases, the convolutional component may comprise a matrix of shape (e.g., with x, y, and z dimensions) where each entry may comprise a feature value at a coordinate of the intermediate output. There may be a number of possible approaches to flattening this output. One approach may comprise flattening the output in its entirety. In some cases, flattening the output in its entirety may complicate learning. While having access to more features for the FCC input, this approach may be less effective than utilizing a pooling layer. The pooling layer may reduce the number of features extracted by averaging over groups of them. This may lose some information, but helps the model avoid overfitting and simplifies the optimization process. In some embodiments, the 2D pooling layer may convert the 7×7×1024 features to a flat vector of 1024 features for the image.

The dropout layer 808 may help prevent overfitting. Dropout layers may allow the network to function with a percentage of connections deactivated. This may be achieved by randomly setting a proportion of inputs of the dropout layer to zero, where the number of zeroed inputs may be determined by the dropout rate. In this way, the network may not base its predictions on a limited number of important features and instead is forced to learn how to use a larger range of features. In an example implementation, the network may use a dropout rate (e.g., at least about 80%, or at least about 90%). In an example embodiment, at least about 95% dropout rate may be used. In some cases, the dropout rate may be used to counter act and/or control overfitting to the training data. In some cases, the dropout layer may be adapted based on the performance observed for the available training data. For example, overfitting may be less problematic for larger training sets, in which case it may be possible to reduce the dropout factor.

The final dense layer 810 may comprise a layer that generates a final score for each class, described elsewhere herein, with the features that have been fed down from the rest of the network. In some cases, for example for the Stage 1 classifier, the final dense layer may map the 1024 output features after 2D average pooling and dropout to six outputs corresponding to the six output classes (three output classes for the Stage 1 classifier, described elsewhere herein). A softmax activation function may be used to convert the network outputs to a probability distribution specifying respective probabilities for each classification. Each output can be interpreted as the prediction probability (or accuracy) of the respective classification (i.e., the probability that the given class is the correct class). The highest-scoring classification may then be selected as the network output for a particular image.

The following hyperparameters (given with default values) may be configured when training a new model: number of dense layers before the output layer (additional layers could be added to increase the model complexity), number of outputs for each layer, number of activation nodes for each layer, dropout rate (e.g., a default value of 0.95), or any combination thereof.

The following additional training parameters (e.g., provided with default values) may also be configured when training a model. In some cases, the training parameters may comprise inputs to the training process rather than values defining the network configuration itself. In some cases, the training parameters may comprise: the loss optimizer utilized (e.g., RMSProp), uses learning rates (LR) defined by exponential decay, the initial learning rate (e.g., 0.0003), decay steps (e.g., 90), decay rate (e.g., 0.96), the loss function utilized (e.g., categorical cross entropy), batch size (e.g., 128), epochs (e.g., 64), checkpoints (e.g., monitoring validation accuracy at the end of training that restores model weights to the best observed weights for the highest accuracy during training, or any combination thereof.

The loss optimizer may determine the algorithm used to determine how the loss of a network, derived from the result of applying the loss function on the predicted values from the network, is converted into weight updates during learning. In some cases, the decay may indicate the learning rate that slowly decreases as training continues. The learning rate may be useful, as typically smaller steps need to be taken the closer the algorithm gets to finding an optimal set of weights.

Batch size may refer to the number of images that the model is trained with before the loss optimizer performs an update step on the model's weights. This parameter may be determined based on a trade-off between memory usage, since the batch of images is processed and optimized in memory in a single instance. In some cases, the greater the number of images in a batch may lead to efficient use of the loss optimizer.

### Model Training (714)

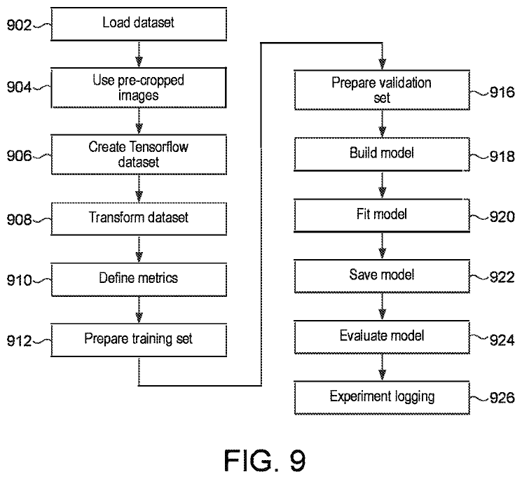

FIG. 9 illustrates the steps in the model training process. In the example implementation described the machine learning system may be based on a pre-existing library (e.g., the TensorFlow™ library) with an in-memory representation of data for the training process.

In some cases, the model training process may comprise a step of loading datasets (902). In some cases, the step of loading a dataset may comprise reading in the dataset (e.g., training and validation CSVs) which define a mapping of image locations in the repository to class labels.

In some cases, the model training process may utilize pre-cropped images 904. In some cases, if pre-cropped images are to be used then this step may update media paths loaded to point to the pre-cropped images in the repository.

In some cases, the training process may comprise creating the pre-existing library dataset (e.g., TensorFlow™ dataset) (906). The dataset may be created using the pre-existing library dataset class (e.g., tf.Dataset class), which may allow a base dataset to be defined to which transformations can be applied to obtain a dataset of uniform shape (e.g., all images having the same size).

In some cases, the training process may comprise transforming the dataset (908). In some cases, transformations may be applied to the images in the dataset, including intensity scaling and/or resizing as described elsewhere herein. This may result in a dataset of images of size 224×224×3.

In some cases, the training process may comprise a step of defining metrics (910). In some cases, the step of defining metrics may define the metrics to track during training, e.g., validation loss and/or validation accuracy.

In some cases, the training process may comprise preparing a training set (912). In some instances, the step of preparing the training set may comprise creating batches, caching the dataset, applying random augmentations (e.g., flipping and/or rotating as described elsewhere herein), or any combination thereof. The system may be configured to pre-fetch the next batch while training on a current batch.

In some cases, the training process may comprise preparing a validation data set (916). In some instances, the step of preparing the validation set may comprise creating a validation set to enable tracking of how the model performs on unseen data during training. The pre-existing library dataset class (e.g., tf.Dataset) can be configured before being passed to the training code, comprising creating batches, caching the data set, configuring the system to pre-fetch the next batch while training on the current batch, or any combination thereof. In some cases, pre-fetching the next batch allows optimum training for the machine learning algorithm, machine learning model, and/or predictive model training, described elsewhere herein, since the training method does not have to wait for data between training cycles.

In some cases, the training process may comprise building a model (918). In some instances, building a model may comprise instantiating a new model using the defined model architecture and hyperparameters, described elsewhere herein. In some cases, the built model may comprise a mobilenet, VGG16, VGG19, ResNet, Inception Net, RetinaNet, Mask R-CNN, or any combination thereof.

In some cases, the training process may comprise fitting a model (920). In some instances, fitting the model may comprise training the model using the training parameters that are provided, resulting in a trained model (with a model version).

In some cases, the training process may comprise saving the model (922). In some instances, saving the model may comprise saving the model to a cloud data repository, using the model version to determine the storage location, together with model metadata (e.g., training parameters used).

In some cases, the training process may comprise evaluating the model (924). In some instances, the model may be evaluated on the validation set. The evaluation results may be stored to the repository at the same location as the model.

In some cases, the training process may comprise logging experiment information (926). In some instances, logging the experiment information may log model performance and metadata (including parameters and model results) to an artificial intelligence, machine learning algorithm, machine learning model, and/or predictive model experimental information repository (e.g., Vertex AI Experiments, and/or Sagemaker). A summary of the model's performance may also be stored to the repository location the model was saved in.

### Model Versioning (716)

Model versions, as described elsewhere herein, may be defined in the form: vX.YYWW.N.K. The vX.YYWW.N portion of the model version may comprise the dataset version that was used for training. The K may comprise the next version number available for this particular version. For example, if v0.2319.2.0 and v0.2319.2.1 already exist, K would be 2. K may be determined by checking the repository location associated with the model results. The next K to assign can be found by checking through the names of the cloud directories that already exist.

### Model Evaluation (718)

Models may be evaluated, as described elsewhere herein, using validation sets. If needed, the hyperparameters and/or training parameters may be modified and the model training process repeated, until the model achieves adequate performance (FIG. 7 loop 712-718). The best performing model may be selected for deployment to a mobile device, described elsewhere herein. For example, models may be periodically updated using the FIG. 7 process as new training data becomes available, and new models are then pushed to the otoscopy application at the mobile devices. The application at each device may store the updated models and uses them for future classification.

### Processing Devices and Systems

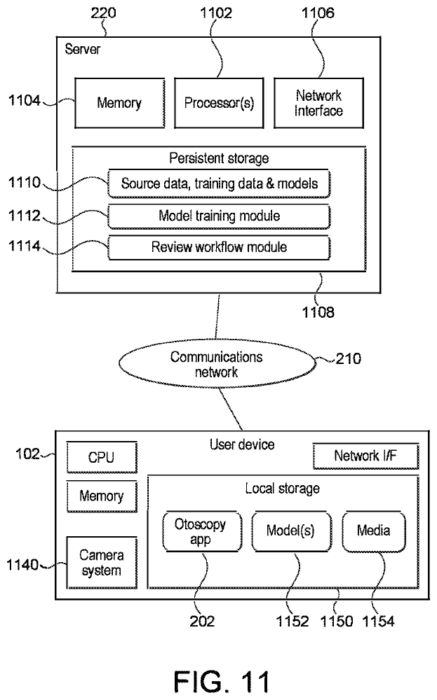

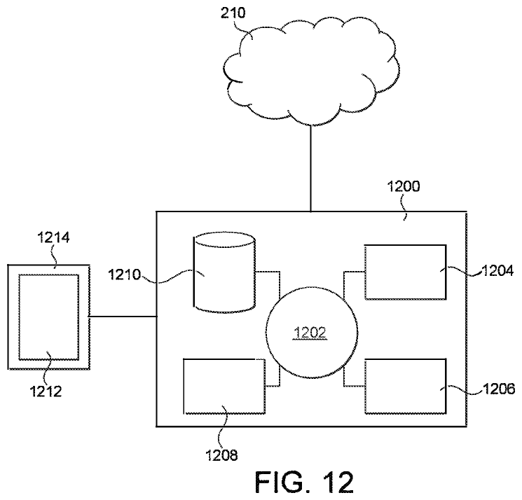

FIG. 11 illustrates example processing devices for implementing described methods, described elsewhere herein.

The server 220 may implement server-side processing functions and may be based on conventional server hardware and as such comprises one or more processors 1102 together with volatile and/or random-access memory 1104 for storing temporary data and software code being executed.

A network interface 1106 may be provided for communication with other system components (e.g., user devices, described elsewhere herein). Communication may occur over one or more networks (e.g., Local and/or Wide Area Networks, including private networks and/or public networks such as the Internet and/or an intranet).

Persistent storage 1108 (e.g., in the form of hard disk storage and/or optical storage) may persistently store software and data for performing various described functions, as described elsewhere herein. In some cases, the persistent storage may store source data, training data, and/or model data 1110, including the clinical data and source images obtained during the subject's appointments. In some cases, the persistent storage may store the data derived from the source data and images. In some cases, the persistent storage may store the classification models. In some instances, the persistent storage may store a model training module 1112 for training the models using the training data. In some cases, the persistent storage may store a review workflow and application module 1114 for managing a review workflow enabling expert practitioners to review images and videos flagged for referral on the user devices.

The persistent storage may further comprise a computer operating system and any other software and data needed for operating the processing device. The device may comprise other hardware components as known to those skilled in the art, where the components may be interconnected by one or more data buses (e.g., a memory bus and I/O bus).

The mobile user device 102 (e.g., a smartphone or similar device) in this example may comprise a standard mobile device hardware platform including CPU, memory, network interface components, or any combination thereof. In some cases, the device may comprise a camera system 1140 e.g., including one or more lenses, CCD sensors, associated control circuitry, or any combination thereof. Permanent local storage 1150 may store local data and software including a mobile device operating system (OS) e.g., iOS and/or Android OS, the mobile otoscopy application 202, local data model(s) 1152 transmitted to the application by the server 220, media (images and/or video) 1154 acquired using the app 202 and classified using the local model(s), or any combination thereof.

While a specific architecture is shown and described by way of example for the server and mobile devices, any combination or arrangement of hardware and/or software architecture may be employed to implement these devices.

Furthermore, functional components indicated as separate may be combined and vice versa. For example, the various functions of the server 220 may be performed by a single server device or may be distributed across multiple devices. For example, model training functions and associated data (1110, 1112) may be hosted on one server (or server cluster), while the review workflow application (1114) may be hosted on another server (or server cluster). In some cases, where the system, described elsewhere herein, may be implemented on a cloud platform (e.g., GCP), functionality may be distributed over a number of server devices and the precise locations where data and software may be hosted may not be predetermined but may be determined as needed by the cloud platform.

The present disclosure provides computer systems that are programmed to implement computer-implemented methods of the disclosure, described elsewhere herein. FIG. 12 shows an exemplary computer system 1200 that may be programmed or otherwise configured to process, analyze, label, view, review, and/or classify one or more images and/or one or more video segments or clips of a subject's ear, as described elsewhere herein. The computer system 1200 can be an electronic device of a user or a computer system that is remotely located with respect to the electronic device. The electronic device can be a mobile electronic device 102, described elsewhere herein.

The computer system 1200 may comprise a central processing unit (CPU, also “processor” and “computer processor” herein) 1202, which can be a single core or multi core processor, or a plurality of processors for parallel processing. The computer system 1200 may also include memory or a memory location 1208 (e.g., random-access memory, read-only memory, and/or flash memory), electronic storage unit 1210 (e.g., hard disk), communication interface 1204 (e.g., network adapter) for communicating with one or more other systems, peripheral devices 1206, such as cache, other memory, data storage and/or electronic display adapters, or any combination thereof. The memory 1208, storage unit 1210, interface 1204 and/or peripheral devices 1206 may be in communication with the CPU 1202 through a communication bus (solid lines, as shown in FIG. 12), e.g., as electrical traces on a motherboard. The storage unit 1210 can be a data storage unit (or data repository) for storing data of one or more images and/or one or more video segments or clips of one or more subjects' ears. The computer system 1200 can be operatively coupled to a computer network (“network”) 210 with the aid of the communication interface 1204. The network 210 can be the Internet, an internet and/or extranet, or an intranet and/or extranet that is in communication with the Internet. The network 210 in some cases may be a telecommunication and/or data network. The network 210 can include one or more computer servers, which can enable distributed computing, such as cloud computing. The network 210, in some cases with the aid of the computer system 1200, can implement a peer-to-peer network, which may enable devices coupled to the computer system 1200 to behave as a client or a server.

The CPU 1202 can execute a sequence of machine-readable instructions, which can be embodied in a program, software, and/or application, described elsewhere herein. The instructions may be stored in a memory location, such as the memory 1208. The instructions can be directed to the CPU 1202, which can subsequently program or otherwise configure the CPU 1202 to implement computer-implemented methods of the present disclosure. Examples of operations performed by the CPU 1202 can include fetch, decode, execute, and writeback.

The CPU 1202 can be part of a circuit, such as an integrated circuit. One or more other components of the system 1200 can be included in the circuit. In some cases, the circuit is an application specific integrated circuit (ASIC).

The storage unit 1210 can store files, such as drivers, libraries and saved programs. The storage unit 1210 can store user data, e.g., one or more images and/or one or more video segments or clips, user preferences, user programs, or any combination thereof. The computer system 1200 in some cases can include one or more additional data storage units that are external to the computer system 1200, such as located on a remote server that is in communication with the computer system 1200 through an intranet or the Internet.

The computer system 1200 can communicate with one or more remote computer systems through the network 210. For instance, the computer system 1200 can communicate with a remote computer system of a user (e.g., personal computing laptop, tablet, and/or desktop system or device). Examples of remote computer systems and/or devices may comprise personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1200 via the network 210.

Computer-implemented methods as described herein can be implemented by way of machine (e.g., computer processor) executable code stored on an electronic storage location of the computer system 1200, such as, for example, on the memory 1208 or electronic storage unit 1210. The machine executable and/or machine-readable code can be provided in the form of software, an application, and/or a mobile smartphone app. During use, the code can be executed by the processor 1202. In some cases, the code can be retrieved from the storage unit 1210 and stored on the memory 1208 for ready access by the processor 1202. In some situations, the electronic storage unit 1210 can be precluded, and machine-executable instructions are stored on memory 1208.

The code can be pre-compiled and configured for use with a machine having a processer adapted to execute the code and/or can be compiled during runtime. The code can be supplied in a programming language that can be selected to enable the code to execute in a pre-compiled or as-compiled fashion.

Aspects of the systems and methods provided herein, such as the computer system 1200, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, and/or flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software and/or application may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and/or electromagnetic waves, such as used across physical interfaces between local devices, through wired and optical landline networks and/or over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” may refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media may comprise, for example, optical or magnetic disks, such as any of the storage devices in any computer(s) or the like, such as may be used to implement the databases, etc. described elsewhere herein and shown throughout the Figures. Volatile storage media may comprise dynamic memory, such as main memory of such a computer platform. Tangible transmission media may include coaxial cables; copper wire and/or fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore may include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, any other medium from which a computer may read programming code and/or data, or any combination thereof. Many of these forms of computer readable media may be involved in carrying one or more sequences of one or more instructions to a processor for execution.

The computer system 1200 can include and/or be in communication with an electronic display 1214 that comprises a user interface (UI) 1212 for providing, for example, an interface for users (e.g., health care professionals, attending physicians, ear nose and throat physicians, physician assistance, registered nurse, or any combination thereof) to review, analyze, process, label, and/or classify one or more images and/or one or more video segments or clips of one or more subjects' ear anatomical features and/or structures e.g., ear canal, tympanic membrane, inner ear, or any combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1202. The algorithm can, for example, classify one or more images and/or one or video segments or clips into one or more categorical classifications, described elsewhere herein.

As used in this specification and the appended claims, the terms “artificial intelligence,” “artificial intelligence techniques,” “artificial intelligence operation,” and “artificial intelligence algorithm” generally refer to any system or computational procedure that may take one or more actions that simulate human intelligence processes for enhancing or maximizing a chance of achieving a goal. The term “artificial intelligence” may include “generative modelling,” “machine learning” (ML), or “reinforcement learning” (RL).

As used in this specification and the appended claims, the terms “machine learning,” “machine learning techniques,” “machine learning operation,” and “machine learning model” generally refer to any system or analytical or statistical procedure that may progressively improve computer performance of a task. In some cases, ML may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

Training the ML model may include, in some cases, selecting one or more untrained data models to train using a training data set. The selected untrained data models may include any type of untrained ML models for supervised, semi-supervised, self-supervised, unsupervised machine learning, and/or transfer learning. The selected untrained data models may be specified based upon input (e.g., user input) specifying relevant parameters, as described elsewhere herein, to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

The systems, the methods, the computer-readable media, and/or the techniques disclosed herein may implement one or more computer vision techniques. Computer vision is a field of artificial intelligence that uses computers to interpret and understand the visual world at least in part by processing one or more digital images from cameras and videos. In some instances, computer vision may use deep learning models (e.g., convolutional neural networks). Bounding boxes may be used in object detection techniques within computer vision. Bounding boxes may be annotation markers drawn around objects in an image. Bounding boxes, are often, although not always, may be rectangularly shaped. Bounding boxes may be applied by humans to training data sets. However, bounding boxes may also be applied to images by a trained machine learning algorithm and/or model that is trained to detect one or more different objects (e.g., humans, hands, faces, cars, etc.). In some cases, bounding boxes detection and tracking techniques may use any object detection annotation techniques, such as semantic segmentation, instance segmentation, polygon annotation, non-polygon annotation, landmarking, 3D cuboids, etc.

In some cases, the machine learning model may implement support vector machine learning techniques. In machine learning, support vector machines (SVMs) may be supervised learning models with associated learning algorithms that analyze data for classification and regression analysis. SVMs may be a robust prediction method, being based on statistical learning. SVMs may be well-suited for domains characterized by the existence of large amounts of data, noisy patterns, or the absence of general theories.

In general terms, SVMs may map input vectors into high dimensional feature space through non-linear mapping function, chosen a priori. In this high dimensional feature space, an optimal separating hyperplane may be constructed. The optimal hyperplane may then be used to determine things such as class separations, regression fit, or accuracy in density estimation. More formally, a SVM constructs a hyperplane or set of hyperplanes in a high or infinite-dimensional space, which can be used for classification, regression, or other tasks like outlier detection.

Support vectors may be defined as the data points that lie closest to the decision surface (or hyperplane). Support vectors may therefore be the data points that are most difficult to classify and may have direct bearing on the optimum location of the decision surface. Given a set of training examples, each marked as belonging to one of two categories, an SVM training algorithm may build a model that assigns new examples to one category or the other, making it a non-probabilistic binary linear classifier (although methods such as Platt scaling exist to use SVM in a probabilistic classification setting). SVM may map training examples to points in space so as to maximize the width of the gap between the two categories. New examples may then be mapped into that same space and predicted to belong to a category based on which side of the gap they fall. In addition to performing linear classification, SVMs can efficiently perform a non-linear classification using what is called the kernel trick, implicitly mapping their inputs into high-dimensional feature spaces.

Within a support vector machine, the dimensionally of the feature space may be large. For example, a fourth-degree polynomial mapping function may cause a 200-dimensional input space to be mapped into a 1.6 billionth dimensional feature space. The kernel trick and the Vapnik-Chervonenkis dimension may allow the SVM to thwart the “curse of dimensionality” limiting other methods and effectively derive generalizable answers from this very high dimensional feature space. Accordingly, SVMs may assist in discovering knowledge from vast amounts of input data.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### EXAMPLES

### Example 1: Model Evaluation Results

In some cases, models were trained on training sets derived using the methodology, described elsewhere herein, from a set of input images and associated classification labels assigned by expert labelers. Evaluation results were then obtained by applying the models to the validation datasets (alternatively, a held-out test set could be used for final evaluation of the models).

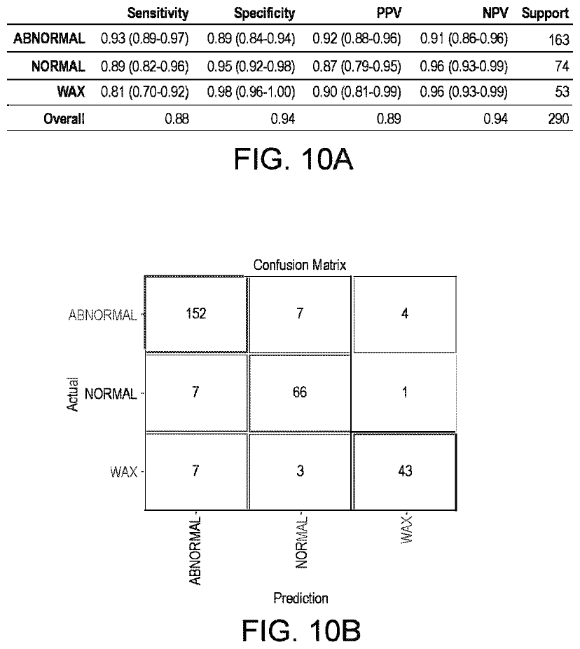

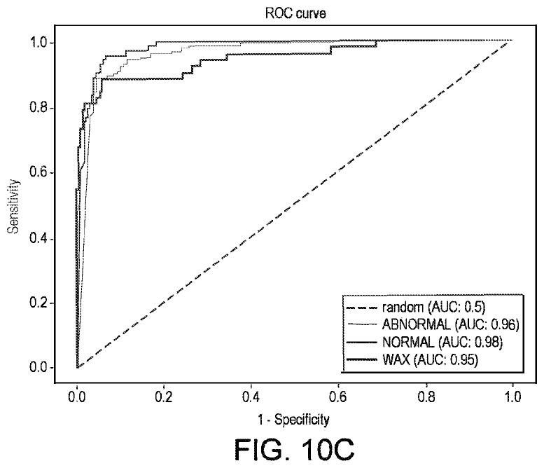

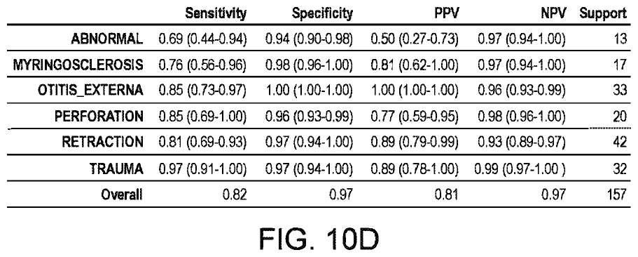

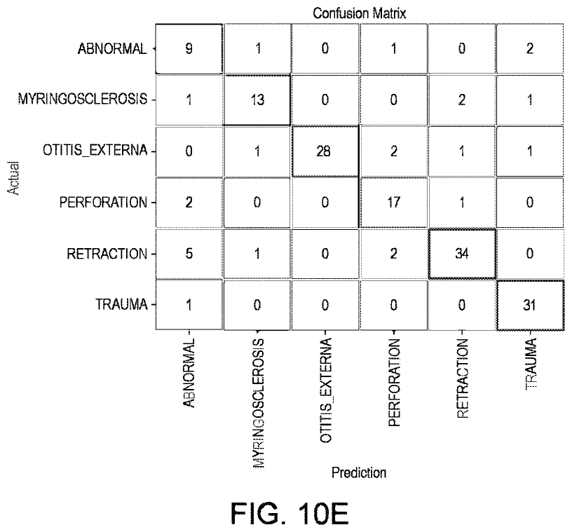

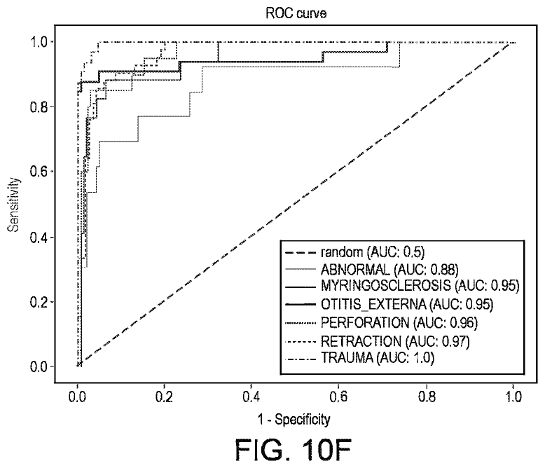

FIG. 10A shows various performance metrics for the model trained for the Stage 1 classifier (predicting three classes: Normal, Wax, and Abnormal). 95% confidence intervals are shown where applicable; support refers to the number of examples for a class. FIG. 10B shows a confusion matrix for the model's predictions. Cells on the diagonal represent correct predictions (classifications), cells off the diagonal represent incorrect predictions. FIG. 10C shows receiver operating characteristic (ROC) curves for each class, with the area under curve (AUC) score shown in the plot legend. In general, the closer the AUC score is to 1, the better the model.

FIG. 10D shows various performance metrics for the model trained for the Stage 2 classifier (predicting five diagnostic classifications plus the uncertain generic “abnormal” classification, described elsewhere herein). A 95% confidence interval is shown where applicable; support refers to the number of examples for a class. FIG. 10E shows a confusion matrix for the model's predictions. Cells on the diagonal represent correct predictions, cells off the diagonal represent incorrect predictions. FIG. 10F shows ROC curves for each class, with the AUC score shown in the plot legend. In general, the closer the AUC score is to 1, the better the model.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount, in some cases near the stated amount by 10%, 5%, or 1%, including increments therein, and in some cases, in reference to a percentage, refers to an amount that is greater or less the stated percentage by 10%, 5%, or 1%, including increments therein.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C”, “one or more of A, B, or C” and “A, B, and/or C” means A alone, B alone, C alone, A and B together, A and C together, B and C together, or A, B and C together.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### CLAUSES

Clause 1. A computer readable medium storing software code for implementing an otoscopy application for processing otoscopy image data, the otoscopy application comprising:
an imaging module configured to acquire image data of a subject's ear canal using a camera system of a mobile device;
an image analysis module configured to process the image data using a trained machine learning model, wherein the trained machine learning model is configured to generate classification data for the image data, the classification data distinguishing between at least a normal classification indicating that the image data is representative of a healthy ear and one or more abnormal classifications relating to abnormal conditions of the ear; and
an upload module configured to transmit the image data and the classification data over a network to a remote review system.

Clause 2. The computer readable medium according to clause 1, wherein the machine learning model comprises:
a first classifier adapted to output an initial classification distinguishing between at least normal and abnormal classifications; and
a second classifier adapted to output one of a plurality of diagnostic classifications corresponding to respective abnormal conditions;
wherein the image analysis module is configured to apply the first classifier to the image data, and to apply the second classifier in response to the first classifier classifying the image data as abnormal, to obtain a diagnostic classification.

Clause 3. The computer readable medium according to clause 1 or 2, wherein the first classifier outputs a classification selected from a set containing at least: a normal class indicating a healthy ear, an abnormal class indicating that an abnormal condition is present, and a wax class indicating that the image data indicates the presence of wax in the ear canal.

Clause 4. The computer readable medium according to clause 2 or 3, wherein the second classifier outputs a classification selected from a set containing at least: a plurality of diagnostic classes for respective individual diagnostic conditions, and a generic abnormal classification for abnormalities not covered by the plurality of individual diagnostic classes.

Clause 5. The computer readable medium according to any of the preceding clauses, wherein the application is further configured to determine a referral indication for the image data in dependence on the classification data to indicate whether the image data should be referred for review, optionally wherein the referral indication indicates referral if the image data is classified as abnormal and non-referral otherwise, wherein the transmitted data includes the referral indication.

Clause 6. The computer readable medium according to any of the preceding clauses, wherein the transmitted data further comprises subject data and/or appointment data relating to the subject.

Clause 7. The computer readable medium according to any of the preceding clauses, wherein the image data comprises at least one of:
one or more images; and
video data, wherein the application is configured to apply the machine learning model to one or more frames of the video data.

Clause 8. The computer readable medium according to any of the preceding clauses, the application configured to:
apply an image classifier to a series of frames of video to obtain a time series of classification values corresponding to respective frames of the video; and
apply a smoothing operation to the time series of classification values to obtain classifications for the one or more frames, plurality of frames, and/or groups of frames.

Clause 9. The computer readable medium according to clause 7 or 8, wherein the smoothing operation comprises a window function applied to successive windows of the classification values, wherein the window function comprises a median window function or other averaging window function; wherein the smoothing operation determines a representative value for each window that is used as the classification value for the frames in the window.

Clause 10. The computer readable medium according to any of clauses 7 to 9, wherein the application is configured to:
classify frames of a video clip as normal or abnormal using a first classifier, and determine a classification of the video clip as normal or abnormal based on the classifications of the frames, wherein the video clip is classified as abnormal if any frame of the video clip was classified as abnormal; and/or
classify frames of the video clip in accordance with a plurality of diagnostic classes using a second classifier, and determine a representative diagnostic classification of the video clip based on the diagnostic classes assigned to the frames, wherein the representative diagnostic classification corresponds to a majority classification of a set of classifications determined for the frames, optionally wherein only classifications assigned a class probability by the second classifier that meets a probability threshold are used in determining the representative or majority classification.

Clause 11. The computer readable medium according to any of clauses 7 to 10, wherein the application is configured to include location data in the transmitted classification data indicating one or more locations of a frame in the video where the frame was classified as abnormal or as associated with a diagnostic classification.

Clause 12. The computer readable medium according to any of the preceding clauses, wherein the machine learning model comprises one or more neural networks implementing one or more image classifiers, optionally implementing the first and/or second classifiers as set out in any of claim 2 to 4 or 10.

Clause 13. The computer readable medium according to clause 12, wherein the machine learning model comprises a neural network which includes:
a feature extraction subnetwork configured to receive a representation of an input image and output a plurality of features derived from the input image;
a dropout layer configured to receive inputs based on outputs from the feature extraction subnetwork, the dropout layer arranged to selectively deactivate a proportion of the inputs in accordance with a dropout rate; and
a dense layer for generating classification probabilities for each of a set of classifications based on an output of the dropout layer.

Clause 14. The computer readable medium according to clause 13, wherein the feature extraction subnetwork comprises a convolution layer and optionally further comprises an average pooling layer operating on outputs of the convolution layer.

Clause 15. The computer readable medium according to any of the preceding clauses, wherein the application is configured to select one or more images or video clips to be uploaded to a remote review system for review by a reviewing user in dependence on the classification data generated for the images or video clips by the machine learning model or based on a referral indication generated in dependence on the classification data, optionally wherein the application is configured to upload an image or video clip in response to the image or video clip being classified as abnormal by the machine learning model.

Clause 16. The computer readable medium according to any of the preceding clauses, wherein the application comprises a user interface configured to display acquired image data, wherein the application is configured to display an indication on the user interface to indicate that displayed image data has been classified as abnormal by the machine learning model.

Clause 17. The computer readable medium according to any of the preceding clauses, wherein the application is configured, in response to a user command to acquire an image, to:
capture an image and display the image on the user interface;
apply the machine learning model to the image; and
in response to obtaining an abnormal classification for the image, display an indication of a potential detected abnormality on the user interface.

Clause 18. The computer readable medium according to any of the preceding clauses, wherein the application is configured, in response to a user command to acquire video data, to:
commence recording video and display the video on the user interface as it is being recorded;
applying the machine learning model to one or more frames of the video; and
in response to obtaining an abnormal classification for a frame of the video, display an indication of a potential detected abnormality on the user interface.

Clause 19. The computer readable medium according to clause 17 or 18, wherein the application is configured to maintain the displayed indication for a predetermined duration after detection of an abnormal frame or after completion of the image or video acquisition and then to remove the indication.

Clause 20. The computer readable medium according to any of the preceding clauses, wherein the application is configured to display during acquisition of image data an indication on the user interface to indicate that acquired images or video are being processed by the machine learning model.

Clause 21. The computer readable medium according to any of the preceding clauses, wherein the machine learning model is applied to one or more frames of the video while recording the video.

Clause 22. The computer readable medium according to any of the preceding clauses, wherein the trained machine learning model is stored at the mobile device.

Clause 23. The computer readable medium according to any of the preceding clauses, wherein the application is further configured to determine a recommendation indication in dependence on the classification data to indicate whether the subject should receive a recommendation for action, optionally wherein the recommendation indication indicates recommendation if the image data is classified as abnormal and non-recommendation otherwise, wherein the transmitted data includes the recommendation indication.

Clause 24. The computer readable medium according to any of the preceding clauses, wherein the application is further configured to determine a recommendation indication in dependence on the referral indication to indicate whether the subject should receive a recommendation for action, optionally wherein the recommendation indication indicates recommendation if the referral indication indicates referral and non-recommendation otherwise, wherein the transmitted data includes the recommendation indication.

Clause 25. The computer readable medium according to any of the preceding clauses, wherein the recommendation for action is a patient group directive or other treatment recommendation.

Clause 26. A mobile device comprising a camera and a computer readable medium storing an application as set out in any of clauses 1 to 25, optionally in combination with an otoscope attachment for the mobile device, the attachment including a speculum and means for projecting an image from the speculum onto the camera of the mobile device.

Clause 27. A computer-implemented method for processing otoscopy image data, comprising:
receiving image data of a subject's ear canal;
processing the image data using a trained machine learning model, wherein the trained machine learning model comprises:
   a first classifier adapted to output an initial classification distinguishing between at least a normal classification indicating that the image data is representative of a healthy ear and an abnormal classification indicative of presence of an abnormal condition of the ear; and
   a second classifier adapted to output one of a plurality of diagnostic classifications corresponding to respective abnormal condition;
wherein the processing step comprises:
   applying the first classifier to the image data to obtain a first classification result; and
   applying the second classifier in response to the first classifier classifying the image data as abnormal to obtain a second classification result indicating a diagnostic classification for the image data;
the method further comprising outputting the first and second classification results.

Clause 28. The method computer-implemented method of clause 27, wherein:
the first classifier outputs a classification selected from a set containing at least: a normal class indicating a healthy ear, an abnormal class indicating that an abnormal condition is present, and a wax class indicating that the image data indicates the presence of wax in the ear canal; and/or
the second classifier outputs a classification selected from a set containing at least: a plurality of diagnostic classes for respective individual diagnostic conditions, and a generic abnormal classification for abnormalities not covered by the plurality of individual diagnostic classes.

Clause 29. The computer-implemented method of clauses 27 or 28, wherein the image data comprises video, the method comprising applying the first and second classifiers to frames of the video, comprising for the first and/or second classifier:
applying the classifier to a series of frames of the video to obtain a time series of classification values corresponding to respective frames of the video; and
applying a smoothing operation to the time series of classification values to obtain classifications for one or more frames, a plurality of frames, and/or groups of frames, wherein the smoothing operation optionally comprises a window function applied to successive windows of the classification values, wherein the window function comprises a median or averaging window function.

Clause 30. The computer-implemented method of any one of clauses 27 to 29, wherein the first and/or second classifier comprises a trained neural network.

Clause 31. The computer-implemented method of any one of clauses 27 to 30, further comprising any of the further steps, features or operations as performed by the application embodied in the computer readable medium of any of claims 1 to 25.

Clause 32. A computer-implemented method of processing otoscopy image data, comprising:
receiving, at a server system, otoscopy data including a plurality of media items from otoscopy applications at a plurality of mobile devices, and classification data determined for the media items by the applications using a classification model;
displaying to a reviewing user via a review application interface, one or more media items with the associated classification data;
receiving input from the reviewing user to assign one or more revised classifications to the one or more media items; and
associating the revised classifications with the media items.

Clause 33. The computer-implemented method of clause 32, wherein the media items comprise a video clip, wherein the classification data includes information indicating an abnormal classification assigned to a frame of the video clip and location information indicating a location in the video clip of the frame to which the classification was assigned, the method comprising:
displaying the video clip on a playback interface of the review application; and
providing a user interface element arranged to initiate playback at a playback location determined in dependence on the location associated with the abnormal classification;
optionally wherein the user interface element comprises a marker element associated with a timeline of the playback interface, the marker element marking the location in the video clip of the frame having the abnormal classification on the timeline, the method comprising moving, responsive to a user interacting with the marker element, a playback position of the video to the location or to a point in the video preceding the location by a predetermined lead-in time;
wherein the method optionally comprises providing a plurality of marker elements indicating respective video clip locations corresponding to respective frames classified as abnormal.

Clause 34. A system comprising a computer device having a processor with associated memory, for performing a method according to any of clauses 27 to 33.

Clause 35. A computer program or computer readable medium comprising software code adapted, when executed by a data processing system, to perform a method according to any of clauses 27 to 33.

Clause 36. A method for identifying a physiological state or condition of an ear of a subject, comprising using a camera-enabled electronic device to capture an image or video from the ear of the subject, and processing the image or video to identify the physiological state or condition of the ear of the subject at an accuracy of at least 80%.

Clause 37. The method of clause 36, wherein the processing comprises using a trained machine learning (ML) algorithm to process the image or video.

Clause 38. The method of clause 36 or clause 37, wherein the trained ML algorithm is stored on the camera-enabled electronic device.

Clause 39. The method of clause 36 or clause 37, wherein the trained ML algorithm is stored on a computer system separate from the camera-enabled electronic device.

Clause 40. The method of clause 39, wherein the computer system comprises a cloud-based computer system.

Clause 41. The method of any one of clauses 36-40, wherein processing of the image or video is conducted with a machine learning algorithm.

Clause 42. A method of training a machine learning algorithm, comprising:
receiving a dataset of one or more images, one or more video segments, or a combination thereof, of an ear of a subject and corresponding physiologic state or condition label of the one or more images, the one or more video segments, or a combination thereof;
transforming the dataset to scale and/or resize the dataset;
preparing a training data set and a validation data set from the transformed dataset; and
training the machine learning algorithm with the training data set and the validation data set, wherein the trained machine learning algorithm has an accuracy of at least about 80% when predicting a physiologic state or condition of one or more images, one or more video segments, or a combination thereof, of a subject's ear.

Clause 43. The method of clause 42, wherein the machine learning algorithm comprises a neural network.

Clause 44. The method of clause 42 or 43, wherein the machine learning algorithm comprises a first classifier and a second classifier.

Clause 45. The method of clause 44, wherein the first classifier is configured to classify a subject's one or more images, one or more video segments, or a combination thereof, as abnormal, normal, or wax.

Clause 46. The method of clause 44 or 45, wherein the second classifier is configured to classify a subject's one or more images, one or more video segments, or a combination thereof, as abnormal, myringosclerosis, otitis externa, perforation, retraction, or trauma.

Clause 47. A method for identifying a physiological state or condition of an ear of a subject, comprising:
receiving an image or video of the ear of the subject;
processing the image or video of the ear of the subject with a first machine learning classifier and a second machine learning classifier; and
identifying the physiological state or condition of the ear of the subject based on at least an output of the first machine learning classifier, an output of the second machine learning classifier, or a combination thereof.

Clause 48. The method of clause 47, wherein the first machine learning classifier, the second machine learning classifier, or a combination thereof, is stored on a camera-enabled electronic device.

Clause 49. The method of clause 48, wherein the first machine learning classifier, the second machine learning classifier, or a combination thereof, is stored on a computer system separate from the camera-enabled electronic device.

Clause 50. The method of clause 49, wherein the computer system comprises a cloud-based computer system.

Clause 51. The method of any one of clauses 47-50, wherein the first machine learning classifier is configured to classify the subject's physiological state or condition of the ear as abnormal, normal, or wax.

Clause 52. The method of any one of clauses 47-51, wherein the second machine learning classifier is configured to classify the subject's physiological state or condition of the ear as abnormal, myringosclerosis, otitis externa, perforation, retraction, or trauma.

Clause 53. A method for identifying a physiological state or condition of an ear of a subject, comprising:
processing a plurality of images or one or more video segments and/or clips from the ear of the subject to determine a time series of classification values;
applying a window operation to the time series of classification values; and
identifying the physiological state or condition of the ear of the subject from the windowed time series classification values.

Clause 54. The method of clause 53, wherein the processing comprises using a trained machine learning (ML) algorithm to process the plurality of images, the one or more video segments and/or clips, or a combination thereof.

Clause 55. The method of clause 54, wherein the trained ML algorithm is stored on a camera-enabled electronic device.

Clause 56. The method of clause 55, wherein the trained ML algorithm is stored on a computer system separate from the camera-enabled electronic device.

Clause 57. The method of clause 56, wherein the computer system comprises a cloud-based computer system.

Clause 58. The method of any one of clauses 53-57, wherein the window operation comprises a smoothening operation.

Clause 59. The method of any one of clauses 53-58, wherein the physiological state or condition of the ear of the subject is predicted from the windowed time series of classification values.

1. A computer readable medium storing software code for implementing an otoscopy application for processing otoscopy image data, the otoscopy application comprising:
an imaging module configured to acquire image data of a subject's ear canal using a camera system of a mobile device;
an image analysis module configured to process the image data using a trained machine learning model, wherein the trained machine learning model is configured to generate classification data for the image data, the classification data distinguishing between at least a normal classification indicating that the image data is representative of a healthy ear and one or more abnormal classifications relating to abnormal conditions of the ear; and
an upload module configured to transmit the image data and the classification data over a network to a remote review system.

2. The computer readable medium according to claim 1, wherein the machine learning model comprises:
a first classifier adapted to output an initial classification distinguishing between at least normal and abnormal classifications; and
a second classifier adapted to output one of a plurality of diagnostic classifications corresponding to respective abnormal conditions;
wherein the image analysis module is configured to apply the first classifier to the image data, and to apply the second classifier in response to the first classifier classifying the image data as abnormal, to obtain a diagnostic classification.

3. The computer readable medium according to claim 2, wherein the first classifier outputs a classification selected from a set containing at least: a normal class indicating a healthy ear, an abnormal class indicating that an abnormal condition is present, and a wax class indicating that the image data indicates the presence of wax in the ear canal.

4. The computer readable medium according to claim 2, wherein the second classifier outputs a classification selected from a set containing at least: a plurality of diagnostic classes for respective individual diagnostic conditions, and a generic abnormal classification for abnormalities not covered by the plurality of individual diagnostic classes.

5. The computer readable medium according to claim 1, wherein the application is further configured to determine a referral indication for the image data in dependence on the classification data to indicate whether the image data should be referred for review.

6. The computer readable medium according to claim 1, wherein the image data comprises at least one of:
one or more images; and
video data, wherein the application is configured to apply the machine learning model to one or more frames of the video data.

7. The computer readable medium according to claim 1, the application configured to:
apply an image classifier to a series of frames of video to obtain a time series of classification values corresponding to respective frames of the video; and
apply a smoothing operation to the time series of classification values to obtain classifications for the one or more frames, plurality of frames, and/or groups of frames.

8. The computer readable medium according to claim 7, wherein the smoothing operation comprises a window function applied to successive windows of the classification values, wherein the window function comprises a median window function or other averaging window function; wherein the smoothing operation determines a representative value for each window that is used as the classification value for the frames in the window.

9. The computer readable medium according to claim 6, wherein the application is configured to:
classify frames of a video clip as normal or abnormal using a first classifier, and determine a classification of the video clip as normal or abnormal based on the classifications of the frames, wherein the video clip is classified as abnormal if any frame of the video clip was classified as abnormal; and/or
classify frames of the video clip in accordance with a plurality of diagnostic classes using a second classifier, and determine a representative diagnostic classification of the video clip based on the diagnostic classes assigned to the frames, wherein the representative diagnostic classification corresponds to a majority classification of a set of classifications determined for the frames.

10. The computer readable medium according to claim 6, wherein the application is configured to include location data in the transmitted classification data indicating one or more locations of a frame in the video where the frame was classified as abnormal or as associated with a diagnostic classification.

11. The computer readable medium according to claim 1, wherein the machine learning model comprises one or more neural networks implementing one or more image classifiers, wherein the machine learning model comprises a neural network which includes:
a feature extraction subnetwork configured to receive a representation of an input image and output a plurality of features derived from the input image;
a dropout layer configured to receive inputs based on outputs from the feature extraction subnetwork, the dropout layer arranged to selectively deactivate a proportion of the inputs in accordance with a dropout rate; and
a dense layer for generating classification probabilities for each of a set of classifications based on an output of the dropout layer.

12. The computer readable medium according to claim 1, wherein the application is configured to select one or more images or video clips to be uploaded to a remote review system for review by a reviewing user in dependence on the classification data generated for the images or video clips by the machine learning model or based on a referral indication generated in dependence on the classification data.

13. The computer readable medium according to claim 1, wherein the application comprises a user interface configured to display acquired image data, wherein the application is configured to display an indication on the user interface to indicate that displayed image data has been classified as abnormal by the machine learning model.

14. The computer readable medium according to claim 1, wherein the application is configured, in response to a user command to acquire an image or video data, to:
capture an image and display the image on the user interface, or commence recording video and display the video on the user interface as it is being recorded;
apply the machine learning model to the image or to one or more frames of the video; and
in response to obtaining an abnormal classification for the image or for a frame of the video, display an indication of a potential detected abnormality on the user interface.

15. The computer readable medium according to claim 14, wherein the application is configured to maintain the displayed indication for a predetermined duration after detection of an abnormal frame or after completion of the image or video acquisition and then to remove the indication.

16. The computer readable medium according to claim 1, wherein the application is configured to display during acquisition of image data an indication on the user interface to indicate that acquired images or video are being processed by the machine learning model.

17. The computer readable medium according to claim 1, wherein the machine learning model is applied to one or more frames of the video while recording the video and/or wherein the trained machine learning model is stored at the mobile device.

18. The computer readable medium according to claim 1, wherein the application is further configured to determine a recommendation indication in dependence on the classification data and/or the referral indication to indicate whether the subject should receive a recommendation for action, wherein the transmitted data includes the recommendation indication.

19. A mobile device comprising a camera and a computer readable medium storing an application as set out in claim 1.

20. A computer-implemented method for processing otoscopy image data, comprising:
receiving image data of a subject's ear canal;
processing the image data using a trained machine learning model, wherein the trained machine learning model comprises:
a first classifier adapted to output an initial classification distinguishing between at least a normal classification indicating that the image data is representative of a healthy ear and an abnormal classification indicative of presence of an abnormal condition of the ear; and
a second classifier adapted to output one of a plurality of diagnostic classifications corresponding to respective abnormal condition;
wherein the processing step comprises:
applying the first classifier to the image data to obtain a first classification result; and
applying the second classifier in response to the first classifier classifying the image data as abnormal to obtain a second classification result indicating a diagnostic classification for the image data;
the method further comprising outputting the first and second classification results.
